SEQUENCE LISTING (9 sequences)

SEQ ID NO: 1 - DNA, 183 bp, Homo sapiens
  mutation (120)..(120): agat is inserted after nucleotide 120 in the CFTR 1461ins4 mutation
ggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaag

SEQ ID NO: 2 - protein, 61 aa, Homo sapiens
GFGELFEKAKQNNNNRKTSNGDDSLFFSNFSLLGTPVLKDINFKIERGQLLAVAGSTGAGK

SEQ ID NO: 3 - DNA, 600 bp, Homo sapiens
atcttagttttagatcatgtcctctagaaaccgtatgctatataattatgtactataaagtaataatgtatacagtgtaatggatcatgggccatgtgcttttcaaactaattgtacataaaacaagcatctattgaaaatatctgacaaactcatcttttatttttgatgtgtgtgtgtgtgtgtgtgtgtttttttaacagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaaggtagttcttttgttcttcactattaagaacttaatttggtgtccatgtctctttttttttctagtttgtagtgctggaaggtatttttggagaaattcttacatgagcattaggagaatgtatgggtgtagtgtcttgtataatagaaattgttccactgataatttactctagttttttatttcctcatattattttcagtggctttttcttc

SEQ ID NO: 4 - DNA, 604 bp, Homo sapiens
atcttagttttagatcatgtcctctagaaaccgtatgctatataattatgtactataaagtaataatgtatacagtgtaatggatcatgggccatgtgcttttcaaactaattgtacataaaacaagcatctattgaaaatatctgacaaactcatcttttatttttgatgtgtgtgtgtgtgtgtgtgtgtttttttaacagggatttggggaattatttgagaaagcaaaacaaaacaataacaatagaaaaacttctaatggtgatgacagcctcttcttcagtaatttctcacttcttggtactcctgtcctgaaagatagatattaatttcaagatagaaagaggacagttgttggcggttgctggatccactggagcaggcaaggtagttcttttgttcttcactattaagaacttaatttggtgtccatgtctctttttttttctagtttgtagtgctggaaggtatttttggagaaattcttacatgagcattaggagaatgtatgggtgtagtgtcttgtataatagaaattgttccactgataatttactctagttttttatttcctcatattattttcagtggctttttcttc

SEQ ID NO: 5 - DNA, 21 bp, Artificial Sequence
  oligonucleotide primer
gggccatgtgcttttcaaact

SEQ ID NO: 6 - DNA, 20 bp, Artificial Sequence
  oligonucleotide primer
gaactaccttgcctgctcca

SEQ ID NO: 7 - DNA, 27 bp, Artificial Sequence
  oligonucleotide probe
aaccgccaacaactgtcctctttctat

SEQ ID NO: 8 - DNA, 20 bp, Artificial Sequence
  oligonucleotide primer
tggggaattatttgagaaag

SEQ ID NO: 9 - DNA, 23 bp, Artificial Sequence
  oligonucleotide primer
cttccagcactacaaactagaaa

(19) United States
(12) Patent Application Publication
(10) Pub. No.: US 2014/0302501 A1
(43) Pub. Date: Oct. 9, 2014

(54) MUTATION DETECTION IN HIGHLY HOMOLOGOUS GENOMIC REGIONS

(71) Applicant: Canon U.S. Life Sciences, Inc., Rockville, MD (US)

(72) Inventors: Ling Xu, Potomac, MD (US); Renee M. Howell, Rockville, MD (US); Caitlin D. Armstrong, Potomac, MD (US)

(21) Appl. No.: 14/358,858

(22) PCT Filed: Nov. 16, 2012
(86) PCT No.: PCT/US2012/065579

§ 371 Date: May 16, 2014

Related U.S. Application Data
(60) Provisional application No. 61/560,799, filed on Nov. 16, 2011.

(51) Int. Cl. C12Q 1/68

(52) U.S. Cl. CPC C12Q 1/6883
USPC 435/6.11; 435/287.2

(57) ABSTRACT

The present invention relates to methods, kits, primers and probes for use in distinguishing highly homologous genomic regions and detecting variants in a locus of interest. In one aspect, the present invention is useful for distinguishing Exon 9 of the CFTR gene from a large homologous region of chromosome 20 in order to determine the presence of the A455E variant.

### CROSS-REFERENCE TO RELATED APPLICATION

The present application claims the benefit of priority to U.S. provisional patent application Ser. No. 61/560,799, filed on Nov. 16, 2011, the entire disclosure of which is incorporated herein by reference in its entirety.

### BACKGROUND

1. Field of the Invention

The present invention relates to methods, kits, primers, probes, and systems for distinguishing highly homologous genomic regions and detecting variants in a locus of interest. More particularly, aspects of the present invention relate to methods, kits, primers, probes, and systems for using a small amplicon assay in combination with unlabeled probes in conducting a high resolution thermal melting analysis of a biological sample containing a locus of interest in order to detect a disease or disorder-causing variant that is present in the locus of interest which shares high homology with other genomic regions. The present invention also relates to methods of detecting a disease in a patient based on the patient's genotype by determining whether the patient has a disease or disorder-causing variant at a locus of interest on the patient's genome.

2. Description of the Background

Cystic fibrosis is the most common lethal autosomal recessive disorder among the Caucasian population and this disease occurs in 1 in 2,500 Caucasian newborns (Rowntree R K, Harris A. Annals of Human Genetics (2003) 67:471-485) (Kerem B S, Rommens J M, Buchanan J A, Science (1989) 245:1073-1080). Currently, more than 30,000 children and young adults were affected by Cystic Fibrosis in the United States. Since the first mutation, ΔF508, for CF was discovered in 1988 using gene cloning technique (Riordan J R, Rommens J M, Kerem B, et al. Science (1989) 245:1066-73), over 1800 mutations on CFTR (cystic fibrosis transmembrane conductance regulator) gene have been added onto CFTR gene database. Among them, A455E on Exon 9 is the second most common mutation of the CFTR gene on chromosome 7 and affects the NBF-1 (Nucleotide Binding Fold-1) of CFTR gene. This mutation is associated with preserved pancreatic function and residual secretion of chloride across membranes (Gan K H, Veeze H J, yen den Ouweland A M W, et al. N Engl J Med (1995) 333:95-99). Early diagnosis of A455E mutation could prevent and lessen the progress of pulmonary and/or pancreatic diseases. A simple accurate and reliable genotype detection is definitively in a great demand to fulfill this purpose.

Traditional sequencing technique has been used to identify and characterize the mutations on CFTR gene (Riordan J R, Rommens J M, Kerem B, et al. Science (1989) 245: 1066-73) (Chu C S, Trapnell B C, Murtagh J J, The EMBO Journal (1991) 10(6):1355-1363). Classical Chromosome walking and jumping techniques were used to identify and sequence of CFTR gene (Rommens J M, Iannuzzi M C, Kerem B, et al. Science (1989) 245 (49:1059-73). These methods included chromosome jumping from the flanking markers, cloning of DNA segments from the defined region with pulsed field electrophoresis, a combination of somatic cell hybrid and molecular cloning that are designated to isolate DNA fragment near CFTR gene, and saturation cloning of a large number of DNA markers from 7q31 region on chromosome 7. These techniques provided the clear and accurate gene map for CFTR gene. Though the complicated procedures and time-labor consuming of these techniques limited their usage for a quick, simple and accurate genotyping in clinical diagnosis, the accuracy and reliable information obtained from these techniques still guide many researchers on exploring CF mutations.

High resolution melting analysis (HRMA), a well established post-PCR amplicon melting based genetic detection method, has been used in screening and genotyping CF mutations. HRMA powers the sensitivity and specificity in identifying and characterizing CF mutations (Chou L S, Lyon E, Wittwer C, Am J Clin Pathol (2005) 124:330-338). The small amplicon method has been applied to HRMA to increase the sensitivity and specificity of HRMA (Liew M, Pryor R, Palais R, et al. Clin Chem (2004) 50:1156-1164). HRMA is the method wherein a rapid post-PCR melting analysis of PCR products occurs immediately after real-time PCR with a saturating dye prior to PCR reaction. The sensitivity of HRMA for mutation scanning can reach to 100% and the specificity of genotyping can be increased with small amplicon melting, unlabeled probe or snackback primers (Wittwer C. Human Mutation (2009) 30:857-859). This method has been widely applied in genetic research and clinical applications.

The Small Amplicon method has been entrenched with HRMA in mutation scanning and genotyping detection in genetic research and diagnosis. Small amplicons, typically smaller than 100 pb, have been proven for displaying high sensitivity and specificity on gene scanning and genotyping (Erali M, Wittwer C T, Methods (2010) 50:250-261) (Wittwer C. Human Mutation (2009) 30:857-859). Most single base mutation within small amplicons can be easily genotyped by high resolution melting. It is noticeable that the advantages of small amplicons are that PCR is efficient and the melting temperatures (Tms) of homozygotes are often separated by approximately 1° C. or greater. However, it is hard to genotype small insertions or deletions with the small amplicon method because the Tm differences between homozygotes could be very small in these circumstances.

### SUMMARY

In one aspect, the present invention provides a method for distinguishing highly homologous genomic regions and detecting variants in a locus of interest. In one embodiment, the method comprises: (a) providing an aliquot of a nucleic acid having a locus of interest; (b) incubating the aliquot of the nucleic acid with a limiting primer, an excess primer, and a probe that is designed to hybridize to the locus of interest on a target strand of the nucleic acid; (c) performing asymmetric PCR using the aliquot to produce an excess of amplicons corresponding to the target strand to which the probe hybridizes, thereby producing a probe-amplicon element; (d) generating a melting curve for the probe-amplicon element in a mixture with a saturating binding dye by measuring fluorescence from the dye as the mixture is heated; and (e) analyzing the melting curve to detect the presence or absence of a variant in the locus of interest.

In another embodiment, the method steps may include (a) providing an amplicon having a locus of interest; (b) hybridizing an unlabeled probe to the locus of interest to form a probe-amplicon element; (c) generating a melting curve for the probe-amplicon element; and (d) analyzing the melting curve to detect the presence or absence of a variant in the locus of interest. In one embodiment, the melting curve is generated in the presence of an intercalating or saturation dye by measuring the fluorescence as the probe-amplicon element is heated.

In another aspect, the present invention provides a method of detecting a disease or a disorder, or a propensity to develop a disease or a disorder, in a patient based on the patient's genotype and a priori knowledge of disease or disorder-causing variant gene sequences associated with the disease. In one embodiment, the method comprises the steps of (a) obtaining a biological sample from the patient; (b) subjecting a portion of the biological sample to asymmetric PCR involving a limiting primer, an excess primer, and a probe to produce a probe-amplicon element; (c) generating a melting curve by subjecting the probe-amplicon element to high resolution thermal melting analysis; and (d) determining whether the patient has a disease or disorder-causing variant. In one embodiment, the melting curve is analyzed to determine whether the patient has a disease or disorder-causing variant. In another embodiment, the biological sample includes nucleic acid that has a locus of interest. In an additional embodiment, hybridization of the probe to the excess complementary strand is increased once the limiting primer is exhausted. In another embodiment, both probe and amplicon duplexes saturated with dye generate melt regions for both the probe and the amplicon.

In another embodiment, the present invention provides a method of detecting a disease or a disorder, or a propensity to develop a disease or a disorder, in a patient based on the patient's genotype and a priori knowledge of benign and disease or disorder-causing variant gene sequences associated with the disease. In one embodiment, the method comprises (a) obtaining a biological sample from a patient; (b) subjecting the sample to asymmetric PCR to produce an amplicon having a locus of interest; (c) subjecting the amplicon to an unlabeled probe assay to produce a first melting curve; and (d) determining whether the patient has a disease or disorder-causing variant. In one embodiment, the melting curve is analyzed to determine whether the patient has a disease or disorder-causing variant.

In another aspect, the present invention provides a method of detecting a variant on a nucleic acid having a locus of interest by (a) performing asymmetric PCR using a primer pair and an unlabeled probe; (b) generating a melting curve for products produced in the asymmetric PCR using the unlabeled probe in a mixture with a saturating binding dye by measuring fluorescence from the dye as the mixture is heated; and (c) analyzing the melting curve to detect whether a variant is present.

In another aspect, the present invention provides a kit for detecting a variant on a target nucleic acid having a locus of interest and/or detecting a disease in a patient based on the patient's genotype. In one embodiment, the kit comprises an unlabeled probe. In another embodiment, the kit may further comprise primers for amplifying a locus of interest on a target nucleic acid. In one embodiment, the primers are selected to amplify the locus of interest without amplifying other highly homologous genomic regions. In another embodiment, the primers are selected for an asymmetric PCR amplification reaction. In a further embodiment, the kit may also comprise instructions for performing an amplification reaction and/or thermal analysis. The kit may also comprise a dye that distinguishes between double stranded and single stranded nucleic acids. A suitable dye may be an intercalating dye, a saturation dye or a double stranded DNA (dsDNA) binding dye.

In another aspect, the present invention provides a system for detecting a variant on a target DNA having a locus of interest and/or detecting a disease in a patient based on the patient's genotype. In one embodiment, the system according to the present invention may include (a) a microfluidic device having a plurality of sample loading zones, each of the sample loading zones being configured to house a separate asymmetric PCR using a nucleic acid having a locus of interest; (b) a HRMA device, comprising a heating element, a fluorescence excitation light source and a fluorescence collection aperture; and (c) a fluorescence derivative melting curve analysis device configured to analyze melting curves generated by the HRMA device so as to detect a variant on the nucleic acid having a locus of interest. In some embodiments, the HRMA device is configured to thermally melt probe-amplicon elements obtained from asymmetric PCRs in said sample loading zones, and to generate fluorescence derivative melting curves for said probe-amplicon elements.

In some embodiments, the amplicon having a locus of interest is produced by mixing a target nucleic acid having a locus of interest with a first primer and a second primer, where the primers are designed to amplify the target nucleic acid having a locus of interest and to distinguish the locus of interest from highly homologous genomic regions, and amplifying the target nucleic acid having a locus of interest to generate an amplicon having the locus of interest. In other embodiments, the amplicon having a locus of interest is produced using asymmetric PCR which utilizes an excess primer and a limiting primer.

In one embodiment, the excess primer hybridizes to a unique heterologous region in the locus of interest as compared to the other highly homologous genomic regions. In another embodiment, the excess primer is a forward primer. In one embodiment, the limiting primer hybridizes as close as possible to the variant in the locus of interest to minimize amplicon size. In another embodiment, the limiting primer is a reverse primer. In one embodiment, the probe hybridizes to the wild-type sequence. In another embodiment, the probe hybridizes to the variant sequence. In an additional embodiment, the probe can be unlabeled. In an additional embodiment, binding of the probe to the excess complementary strand is increased once the limiting primer is exhausted. In an additional embodiment, both probe and amplicon duplexes saturated with dye generate melt regions for both the probe and the amplicon.

In one embodiment, the locus of interest is highly homologous to one or more genomic regions and the method distinguishes the locus of interest from the other highly homologous genomic regions.

In another embodiment, the locus of interest is on a gene associated with a disease or disorder such as cystic fibrosis. In another embodiment, the locus of interest is exon 9 of the CFTR gene. In an additional embodiment, the variant is the A455E variant in exon 9 of the CFTR gene. In a further embodiment, the variant is the 1461ins4 variant in exon 9 of the CFTR gene.

It is within the scope of an embodiment of the invention to obtain high resolution thermal melting curves using unlabeled probes for target DNA sequences. It is also within the scope of an embodiment of the invention to design probes and primers that are suitable for amplifying the target nucleic acid having a locus of interest and to distinguish the locus of interest from highly homologous genomic regions. In one embodiment, the primers are designed to distinguish between the target nucleic acid and highly homologous genomic regions so that only the target of interest is amplified.

Thus, in another aspect, the present invention also provides a method of designing primers and probes that are useful for thermal melt analysis of a locus of interest that contains a disease or disorder-causing variant on a target nucleic acid that is highly homologous to other genomic regions. In accordance with this aspect, the method comprises (a) selecting a locus of interest of a disease or disorder, in which the locus of interest has a disease or disorder-causing variant and in which the locus of interest is on a nucleic acid that is highly homologous with other genomic regions, (b) designing a pair of primers for use in asymmetric PCR, and (c) designing a probe for hybridizing to one strand of the locus of interest. In one embodiment, one primer is designed to hybridize to a unique heterologous region in the locus of interest as compared to the other highly homologous genomic regions. In another embodiment, this primer is the excess primer. In an additional embodiment, the excess primer is a forward primer. In one embodiment, one primer is designed to hybridize as close as possible to the variant in the locus of interest to minimize amplicon size. In another embodiment, this primer is the limiting primer. In an additional embodiment, the limiting primer is a reverse primer. In one embodiment, the probe hybridizes to the wild-type sequence. In another embodiment, the probe hybridizes to the variant sequence.

The above and other aspects and features of the present invention, as well as the structure and application of various embodiments of the present invention, are described below with reference to the accompanying drawings.

### BRIEF DESCRIPTION OF THE DRAWINGS

The accompanying drawings, which are incorporated herein and form part of the specification, illustrate various embodiments of the present invention.

FIG. 1 shows a DNA sequence BLAST Search Map from NCBI Database. The total DNA sequence is 1140 bp, including 317 bp of intron 8, 183 bp of exon 9 and 640 bp of intron 9 of the CFTR gene.

FIG. 2 shows UCSC Human BLAT Search Scores. The total DNA sequence is 1140 bp, including 317 bp of intron 8, 183 bp of exon 9 and 640 bp of intron 9 of the CFTR gene.

FIG. 3 shows the CFTR exon 9 gene map. The nucleotide (SEQ ID NO:1) and amino acid (SEQ ID NO:2) sequences for exon 9 are shown. All the known variants in the region are displayed graphically underneath. The A455E mutation is circled. Other mutations close to the A455E mutation are also pictured.

FIG. 4 shows BLAST AND BLAT search results for exon 9 of the CFTR gene. The 182 bp of exon 9 sequence was queried using the NCBI BLAST algorithm (top) and UCSC Genome Browser BLAT algorithm (bottom). Both algorithms show the high amount of sequence homology that exists between the CFTR exon 9 sequence and other regions of the human genome.

FIG. 5 shows BLAST AND BLAT search results for exon 9 and adjacent introns of the CFTR gene. A total of 720 bp was queried using the NCBI BLAST algorithm (top) and UCSC Genome Browser BLAT algorithm (bottom). This includes the 183 exon 9 as well as 77 bp from intron 8 and 460 bp from intron 9. Both algorithms show the high amount of sequence homology that exists between exon 9 and its adjacent intronic sequences with other regions of the human genome. The small region (shown on the left side in the top image) which has a unique sequence was the target for primer design.

FIG. 6 shows primer and unlabeled probe design for the CFTR exon 9 A455E mutation. The forward primer was designed in the small region of intron 8 which contains unique genomic sequence. The reverse primer was designed as close as possible to the A455E mutation to minimize amplicon size. As the amplicon size of 304 bp is too large for use in small amplicon genotyping, an unlabeled probe was designed to specifically target the A455E mutation.

FIG. 7 shows a first derivative of the melt profile for CFTR exon 9 A455E. The probe melt is shown on the left, from 60° C. to 74° C. The wild-type DNA displays a single feature, and the heterozygous DNA shows two features. The amplicon melt is shown on the right, from 75° C. to 84° C.

FIG. 8 shows a first derivative of the melt profile for CFTR exon 9 A455E. The probe melt is shown on the left, from 61° C. to 74° C. The wild-type DNA displays a single feature, and the heterozygous DNA shows two features. The homozygous construct DNA shows a single melt feature as well which corresponds to the second melt feature of the heterozygous DNA. The amplicon melt is shown on the right, from 75° C. to 84° C.

FIG. 9 shows a synthetic construct design. The pGOv4 plasmid vector is shown on the left. It contains two antibiotic resistance genes as well as a replication origin. The CFTR exon 9 inserts (wild-type (SEQ ID NO:3) and 1461ins4 homozygous (SEQ ID NO:4) are shown on the right. The 1461ins4 mutation is shown in bold. Exon sequence is denoted by bold capital letters, and intron sequence is denoted by lowercase letters. The annealing sites of the primers described in Table 1 are underlined.

FIG. 10 shows a high resolution melt (HRM) of CFTR exon 9 DNAs with a prior primer design. The melt behavior indicates that more than one PCR product is present in the genomic DNA sample. The true wild-type pattern, as shown by the synthetic construct in dashed lines, is not exhibited by the genomic DNA. It is highly likely that the pseudogene region is amplified in the genomic DNA.

FIG. 11 shows gene scanning of exon 9 DNAs with a prior primer design.

FIG. 12 shows bioanalyzer results with a prior primer design. The results from the wile-type construct DNA is shown on the left, and the wild-type genomic DNA is shown on the right. The construct DNA shows a clean single peak in addition to the two internal size markers used in this assay, which is indicative of a single PCR product. The genomic DNA shows a main peak, with some smaller peaks to the right of the main peak. When these peaks are integrated, they constitute only about 3 ng/μl total. A clear secondary peak is not seen.

FIG. 13 shows a HRM of exon 9 DNAs with a new primer design in accordance with one embodiment of the present invention. Genomic and construct wild-type DNAs show similar melt patters with the new primer designs. The exon 9 pseudogene region is no longer amplified during PCR in addition to the true CFTR exon 9 region.

FIG. 14 shows gene scanning of CFTR exon 9 DNAs with a new primer design in accordance with one embodiment of the present invention. The gene scanning result of the assay is shown in this figure. The difference between the fluorescence signals during the melt transition is shown as a function of temperature. DNAs with the same sequence should cluster together on a difference plot. Here, the construct and the genomic wild-type DNAs do cluster together, and the construct heterozygotes and homozygotes are clearly distinct from both types of wild-type DNAs.

FIG. 15 illustrates a microfluidic device embodying aspects of the present invention.

FIG. 16 is a functional block diagram of a system for using a microfluidic device embodying aspects of the present invention.

FIG. 17 illustrates a microfluidic system embodying aspects of the present invention.

### DETAILED DESCRIPTION OF THE INVENTION

The present invention has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, Oligonucleotide Synthesis: A Practical Approach, 1984, IRL Press, London, Nelson and Cox (2000), Lehninger Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

As used herein, “homozygous” refers to a genotype consisting of two identical alleles at a given locus.

As used herein, “heterozygous” refers to a genotype consisting of two different alleles at a given locus.

As used herein, “variant” refers to a permanent change in the DNA sequence of a gene, including mutations. Variants and mutations in a gene's DNA sequence can alter the amino acid sequence of the protein encoded by the gene.

As used herein, “locus of interest” refers to a sequence of nucleotides on a nucleic acid/amplicon that is to be detected and/or analyzed. The locus of interest can be a site where variants/mutations are known to cause disease or predispose to a disease state. A locus of interest can be a site of targeted nucleic acid variant within the context of a gene. A locus of interest may encompass a genomic sequence that includes the site of a variant.

As used herein, “target nucleic acid” refers to one or more DNA or RNA molecule(s) that is to be replicated, amplified, detected, and/or analyzed. A “target nucleic acid” refers to deoxyribonucleic acid, ribonucleic acid or mixtures thereof. In addition, a “target nucleic acid” can further comprise non-natural nucleic acids. The target nucleic acid can be generated by any number of means. For example, it can be generated from a cleavage reaction by a restriction enzyme or other endo- or exonucleases. Alternatively, it can form as a result of a specific or non-specific cleavage of a longer nucleic acid strand, and can be generated enzymatically or chemically. The target nucleic acid of the present invention also contemplates fragments generated naturally in vivo, by aged tissue, apoptotic cells, or the consequence of any other natural, biological or chemical reaction that may generate nucleic acid fragments. The target nucleic acid may contain the locus of interest as a portion of its sequence or the locus of interest may make up the entire sequence of the target nucleic acid.

As used herein, “highly homologous genomic regions” or “substantially homologous genomic regions” refer to two or more regions of a genome, such as the human genome, that have a very high degree of homology or identity such as at least 85%, or at least 90%, or at least 92%, or at least 95%. In some embodiments, such high homologous genomic regions may include a gene and a pseudogene.

As used herein, “pseudogene” refers to genomic DNA sequences that are similar to normal genes but are non-functional. Pseudogenes are sometimes regarded as defunct relatives of functional genes.

As used herein, “probe-amplicon elements” and “probe/primer amplicon” refer to the nucleic acid fragments or amplicons that are generated during PCR using a primer and a probe.

As provided throughout the specification, the steps of all of the methods described herein can occur in a sequential or simultaneous manner, and alternatively some portion of the steps can occur simultaneously while others occur sequentially.

Some embodiments of the present invention utilize thermal melt curves to distinguish between at least two variants on a target nucleic acid having a locus of interest. Thermal melt curves of fluorescence have been used in the art to determine the melting temperature of a DNA strand when denatured from the duplex state to the two separate single strands via a ramp increase in temperature. Typically, the melting temperature or Tm is defined to be the temperature at which 50% of the paired DNA strands have denatured into single strands. Intercalating dyes that fluoresce when bound to double stranded DNA and lose their fluorescence when denatured are often used in measuring Tm. Typically, the negative derivative of fluorescence with respect to temperature (−dF/dT) has been used in the determination of Tm. In typical systems, the temperature at the peak −dF/dT is used as an estimate of the melting temperature Tm.

Melting curve analysis is typically carried out either in a stopped flow format or in a continuous flow format. In one example of a stopped flow format, melting curve analysis is done in a chamber to which the nucleic acid sample has been added. In an alternative stopped flow format, flow is stopped within a microchannel of a microfluidic device while the temperature in that channel is ramped through a range of temperatures required to generate the desired melt curve. In one example of a continuous flow format, a melting curve analysis is performed by applying a temperature gradient along the length (direction of flow) of a microchannel of a microfluidic device. If the melting curve analysis requires that the molecules being analyzed be subjected to a range of temperatures extending from a first temperature to a second temperature, the temperature at one end of the microchannel is controlled to the first temperature, and the temperature at the other end of the length is controlled to the second temperature, thus creating a continuous temperature gradient spanning the temperature range between the first and second selected temperatures. An example of an instrument for performing a melting curve analysis is disclosed in U.S. Patent Application Publication No. 2007/0231799 and U.S. Patent Application Publication No. 2012/0058571, each of which area incorporated herein by reference in its entirety.

The thermal melt data that is analyzed in accordance with aspects of the present invention is obtained by techniques well known in the art. See, e.g., Knight et al. (U.S. Patent Application Publication No. 2007/0231799); Knapp et al. (U.S. Patent Application Publication No. 2002/0197630); Knight et al. (U.S. Patent Application Publication No. 2012/0058571); Wittwer et al. (U.S. Pat. No. 7,456,281); and Wittwer et al. (U.S. Pat. No. 6,174,670). Although the present invention is applicable to the analysis of thermal melt data obtained in any environment, it is particularly useful for thermal melt data obtained in the microfluidic environment because of the need for greater sensitivity in this environment.

Thermal melt data is typically generated by elevating the temperature of a molecule or molecules, e.g., of one or more nucleic acids, for a selected period of time and measuring a detectable property emanating from the molecule or molecules, wherein the detectable property indicates an extent of denaturation of the nucleic acid. This period of time can range, for example, from about 0.01 second through to about 1.0 minute or more, from about 0.01 second to about 10 seconds or more, or from about 0.1 second to about 1.0 second or more, including all time periods in between. In one embodiment, heating comprises elevating the temperature of the molecule or molecules by continuously increasing the temperature of the molecule or molecules. For example, the temperature of the molecule(s) can be continuously increased at a rate in the range of about 0.1° C./second to about 1° C./second. Alternatively, the temperature of the molecule(s) can be continuously increased at a slower rate, such as a rate in the range of about 0.01° C./second to about 0.1° C./second, or at a faster rate, such as a rate in the range of about 1° C./second to about 10° C./second. The heating can occur through application of an internal or an external heat source, as is known in the art.

The actual detection of a change(s) in a physical property of the molecules can be detected in numerous methods depending on the specific molecules and reactions involved. For example, the denaturation of the molecules can be tracked by following fluorescence or emitted light from molecules in the assay. The degree of, or change in, fluorescence is correlational or proportional to the degree of change in conformation of the molecules being assayed. Thus, in some methods, the detection of a property of the molecule(s) comprises detecting a level of fluorescence or emitted light from the molecules(s) that varies as a function of relative amounts of binding. In one configuration, the detecting of fluorescence involves a first molecule and a second molecule, wherein the first molecule is a fluorescence indicator dye or a fluorescence indicator molecule and the second molecule is the target molecule to be assayed. In one embodiment, the fluorescence indicator dye or fluorescence indicator molecule binds or associates with the second molecule by binding to hydrophobic or hydrophilic residues on the second molecule. The methods of detecting optionally further comprise exciting the fluorescence indicator dye or fluorescence indicator molecule to create an excited fluorescence indicator dye or excited fluorescence indicator molecule and discerning and measuring an emission or quenching event of the excited fluorescence indicator dye or fluorescence indicator molecule. See, e.g., Boles et al. (U.S. Pat. No. 8,145,433); Cao et al. (U.S. Pat. No. 8,180,572); Knight et al. (U.S. Patent Application Publication No. 2007/0231799), which are incorporated herein by reference in their entireties.

Several techniques exist for the measurement of the denaturation of the molecules of interest, and any of these can be used in generating the data to be analyzed in accordance with aspects of the present invention. Such techniques include fluorescence, fluorescence polarization, fluorescence resonance energy transfer, circular dichroism and UV absorbance. Briefly, the fluorescence techniques involve the use of spectroscopy to measure changes in fluorescence or light to track the denaturation/unfolding of the target molecule as the target molecule is subjected to changes in temperature. Spectrometry, e.g. via fluorescence, is a useful method of detecting thermally induced denaturation/unfolding of molecules. Many different methods involving fluorescence are available for detecting denaturation of molecules (e.g. intrinsic fluorescence, numerous fluorescence indicator dyes or molecules, fluorescence polarization, fluorescence resonance energy transfer, etc.) and are optional embodiments of the present invention. These methods can take advantage of either internal fluorescent properties of target molecules or external fluorescence, i.e. the fluorescence of additional indicator molecules involved in the analysis. See, e.g., Cao (U.S. Patent Application Publication No. 2010/0233687), incorporated herein by reference in its entirety.

One method of measuring the degree of denaturation/unfolding of the target molecule is through monitoring of the fluorescence of dyes or molecules added to the microfluidic device along with the target molecule and any test molecules of interest. A fluorescence dye or molecule refers to any fluorescent molecule or compound (e.g., a fluorophore) which can bind to a target molecule either once the target molecule is unfolded or denatured or before the target molecule undergoes conformational change by, for example, denaturing and which emits fluorescent energy or light after it is excited by, for example, light of a specified wavelength.

One dye type typically used in the microfluidic devices is one that intercalates within strands of nucleic acids. An example of such a dye is ethidium bromide. An exemplary use of ethidium bromide for binding assays includes, for example, monitoring for a decrease in fluorescence emission from ethidium bromide due to binding of test molecules to nucleic acid target molecules (ethidium bromide displacement assay). See, e.g., Lee et al. (J Med Chem 36:863-870, 1993). The use of nucleic acid intercalating agents in measurement of denaturation is known to those in the art. See, e.g., Haugland (Handbook of Fluorescent Probes and Research Chemicals, 9^{th} Ed., Molecular Probes, Inc., Eugene, Oreg., 2002).

Dyes that bind to nucleic acids by mechanisms other than intercalation are also typically employed in thermal melt analysis. For example, dyes that bind the minor groove of double stranded DNA can be used to monitor the molecular unfolding/denaturation of the target molecule due to temperature. Examples of suitable minor groove binding dyes are the SYBR® Green family of dyes sold by Molecular Probes Inc. (Eugene, Oreg., USA). See, e.g., Haugland (Handbook of Fluorescent Probes and Research Chemicals, 9^{th} Ed., Molecular Probes, Inc., Eugene, Oreg., 2002). SYBR® Green dyes will bind to any double stranded DNA molecule. When a SYBR® Green dye binds to double stranded DNA, the intensity of the fluorescent emissions increases. As more double stranded DNA are denatured due to increasing temperature, the SYBR® Green dye signal will decrease. Other suitable dyes are LCGreen® Plus sold by Idaho Technology, Inc. (Salt Lake City, Utah, USA), SYTO® 9 sold by Invitrogen Corp. (Carlsbad, Calif.) and, Eva Green® sold by Biotium Inc. (Hayward, Calif.). Further examples of dyes include SYBR® Green I (BIORAD, Hercules, Calif.), ethidium bromide, SYBR® Gold (INVITROGEN), Pico Green, TOTO-1 and YOYO-1. It is within the skill of persons of ordinary skill in the art to select a suitable dye.

In accordance with aspects of the present invention, methods, kits, primers, probes, and systems for distinguishing highly homologous genomic regions and detecting variants in a locus of interest are provided. In one exemplary embodiment, the method of the present invention is useful for detecting the presence or absence of the A455E variant in exon 9 of the CFTR gene.

A DNA sequence of interest is about 640 bp in length, which contains the entire exon 9 of the CFTR gene (183 bp in length), and its intron junctions (about 400 bp on intron 9 junction and about 50 bp on intron 8 junction), are over 96% homologous on the human genome, in particular with the regions on chromosome 20. This nucleotide sequence similarity makes genotyping detections for the mutations on exon 9 more difficult, especially when using a small amplicon method. See, Liew et al. (Liew M, Pryor R, Palais R, et al., Clin Chem (2004) 50:1156-1164), Erali et al. (Erali M, Wittwer C T, Methods (2010) 50:250-261), Wittwer (Wittwer C, Human Mutation (2009) 30:857-859) and Xu et al. (U.S. patent application Ser. No. 13/297,970 filed on Nov. 16, 2011) for a description of the small amplicon methods. Some research studies showed that the large sequence of the CFTR exon 9 and its intron junctions are duplicated especially with a region of chromosome 20, and could affect patients' mutation identifications (El-Speedy, A., Dudognon, T., Bilan, F., et al., J Mol Diag (2009) 11:488-493). Though studies on genotyping mutation A455E have been reported using different detection techniques, no detailed descriptions on how to distinguish this mutation from other homologous sequences on the human genome have been reported. The homologous nature of exon 9 can cause the duplication of pseudogenes. This PCR bias can complicate and even mislead the diagnosis on genotyping.

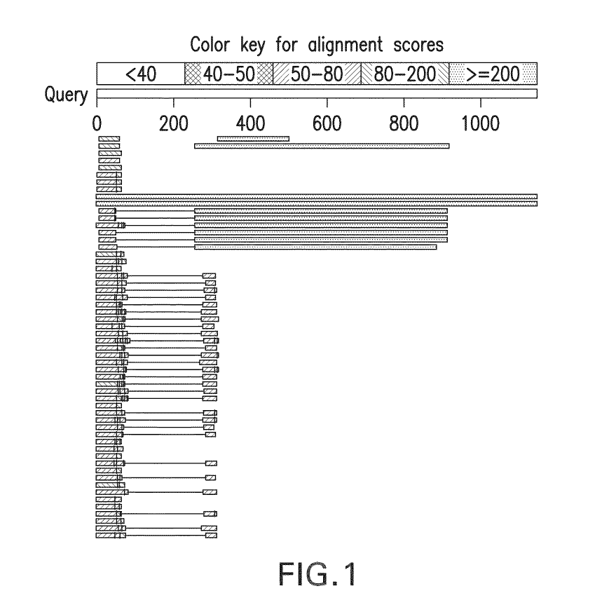

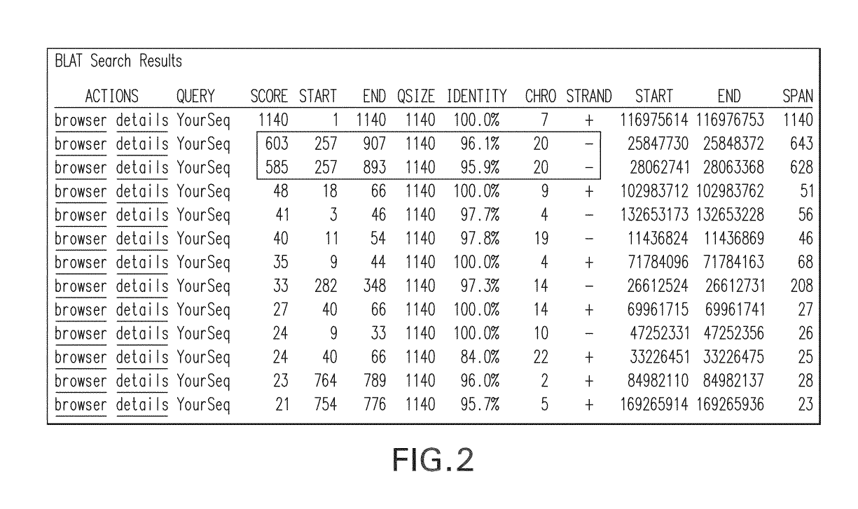

The sequence blast search results obtained from NCBI Blast Database and UCSC Human BLAT SEARCH GENOME database (University of California, Santa Cruz) show that CFTR exon 9 and its intron junctions are highly homologous to other sequences on the human genome, especially on chromosome 20. See, FIGS. 1 and 2.

As shown in FIGS. 1 and 2, a region of about 640 bp, including CFTR intron 8, exon 9 and intron 9, are highly homologous to other genomic regions, mainly to chromosome 20 (about 96%). This region far exceeds the typical size of a small amplicon, i.e., typically smaller than 100 bp.

Though classical chromosome walking and jumping techniques are still standards in identifying the gene sequence, the whole identification process involves several distinct processes, including chromosome jumping from the flanking markers, DNA cloning with pulsed field electrophoresis, a combination of somatic cell hybrid and molecular coning for isolating DNA fragment around CFTR gene, and saturation cloning of a large number of DNA makers in 7q31 region on chromosome 7. These processes require more complicated experimental procedures and the data acquisition and analysis processes is time/labor consuming. It is very difficult to apply these techniques to a quick turn-around clinical routine diagnosis process for CFTR genotyping, and these techniques do not fit the platform of many melting systems.

A small amplicon method with HRMA has the advantage to identify specific targeted mutations. However, this method is limited in its ability to detect mutations that may be found in a region that is largely homologous with other genomic regions and in differentiating the targeted mutation from the homologous regions of different chromosomes, due to the small size of the amplicons it generates. One example of this limitation is shown, for instance, with the A455E variant in exon 9 on chromosome 7 because of the large homologous region that occurs on chromosome 20.

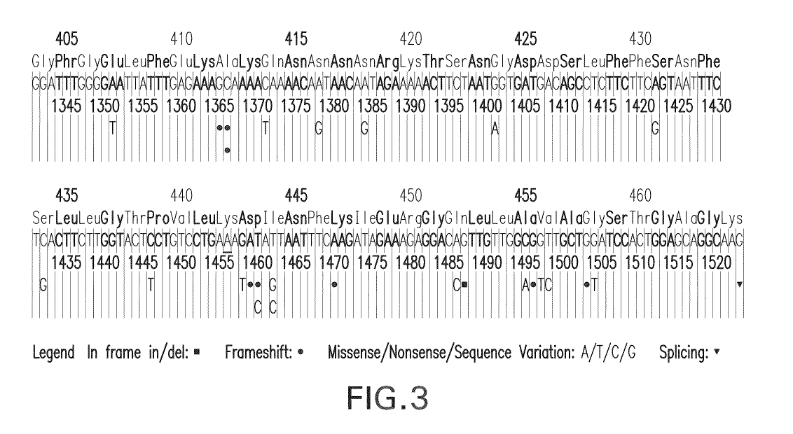

The CFTR gene is located on 7q31.2 of chromosome 7. Exon 9 of the gene is 183 bp in length and contains multiple mutations including A455E (FIG. 3, credit to genet.sickkids.on.ca), a common causative mutation of cystic fibrosis. The A455E mutation is a single base change from C to A and affects the NBF-1 (Nucleotide Binding Fold-1) of the CFTR protein. This mutation is associated with a less severe phenotype than what is observed in patients with the most common ΔF508 mutation, as the CFTR protein retains some functionality with the A455E mutation. Fewer patients exhibit pancreatic insufficiency, and the degree of pulmonary disease is more mild than in patients with the ΔF508 mutation (Gan, K. H. et al., Thorax (1995) 50:1301-1304).

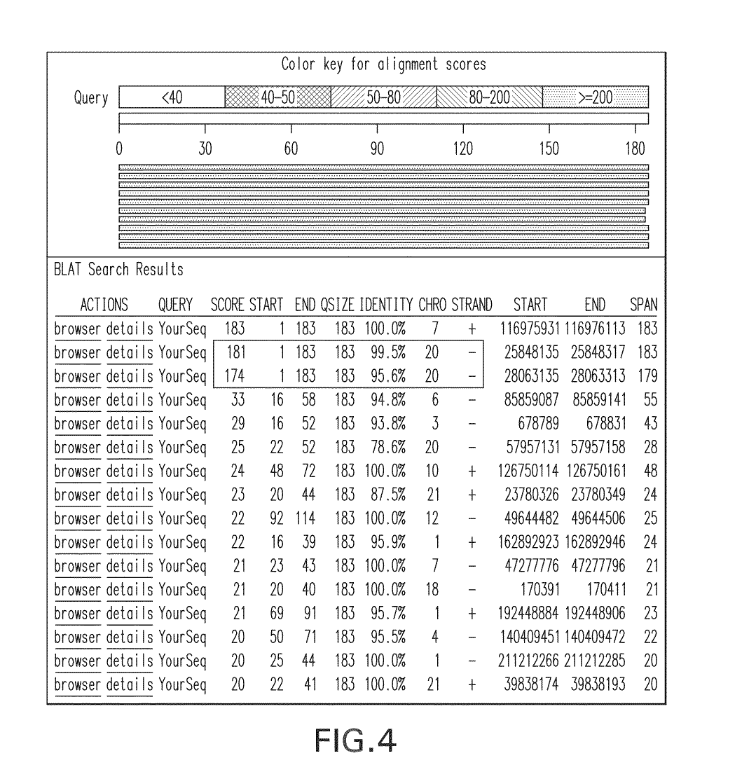

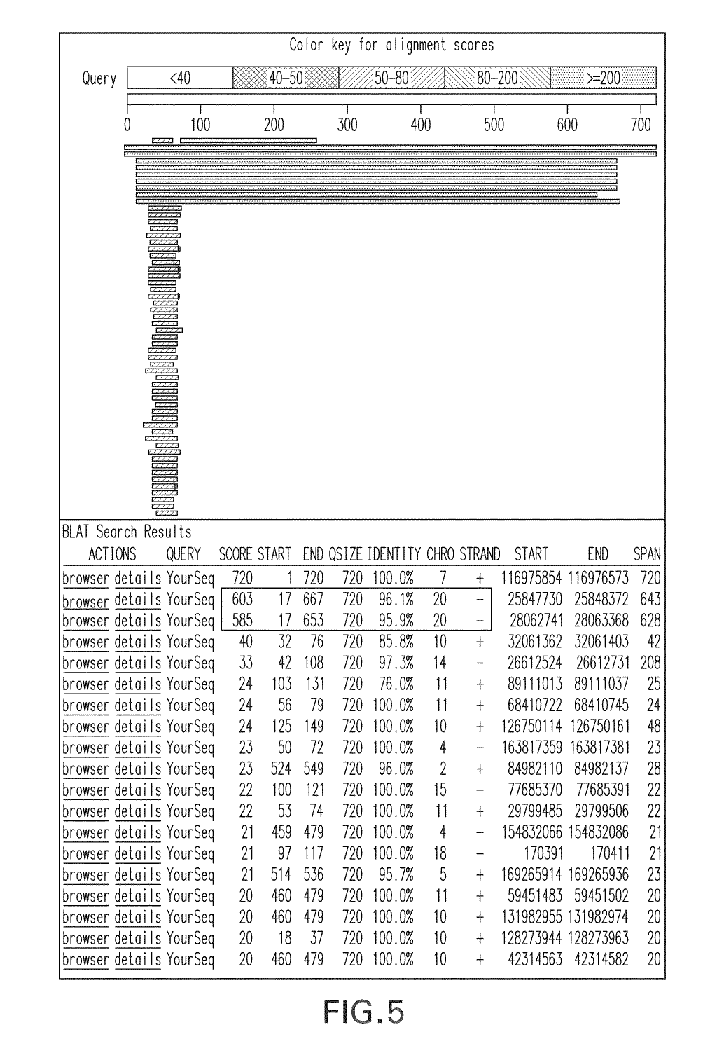

The problem with designing PCR-based assays for the exon 9 region of the CFTR gene is that the majority of the exon and the adjacent intronic sequences share sequence homology with other regions of the genome. In particular, a pseudogene is known to exist on chromosome 20 (Liu, Y et al., Genome Biol (2004) 5:R64). The exon itself is one hundred percent homologous with other genomic areas (FIG. 4) and the majority of the exon's flanking intronic sequence (FIG. 5) share sequence homology with other regions of the genome. The exon 9 intron junctions (intron 8 and intron 9) are also homologous with chromosome 20.

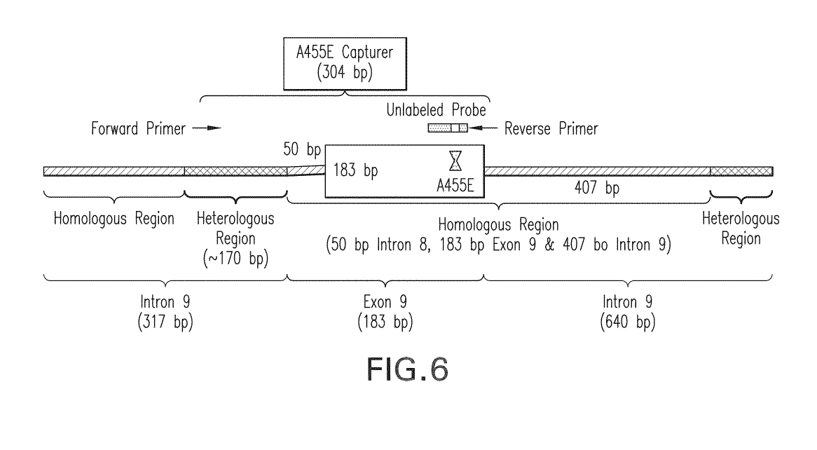

In order to differentiate the CFTR exon 9 from other large homologous regions of different chromosomes, especially chromosome 20, a new approach was devised. In accordance with one embodiment, FIG. 6 details the theory behind the new primer and probe designs which target the exon 9 A455E mutation.

As mentioned previously, both the CFTR exon 9 and flanking intron sequences share sequence homology with other regions of the genome. The exon itself is 183 bp in length and completely homologous to other genomic regions. The sequence homology extends 50 bp upstream of the exon into intron 8. There is then a short (approximately 170 bp) region which contains unique sequence before additional homologous sequence is encountered. In addition, the first 407 bp of intron 9 just downstream of exon 9 shares sequence homology with other regions of the genome. If primers were designed completely outside of the homologous region, the resulting PCR product would be over 1000 bp long. That is too long for any real utility in PCR-based genotyping assays. In accordance with certain embodiments of the present invention, it was decided that the forward primer would be designed within the unique region of intron 8 and as a compromise the reverse primer would be designed within the region of the exon that shares sequence homology with other parts of the genome.

In this approach, the forward primer is placed in the heterologous region in CFTR intron 8, and the reverse primer is placed close to the exon 9 A455E mutation in intron 9 to make the amplicon size as small as possible. An unlabeled probe is used to include the A455E mutation. The purpose of this design is to distinguish exon 9 from the large homologous region on chromosome 20 using the forward primer, and to characterize the A455E mutation using the unlabeled probe.

The amplicon size obtained from this design is about 304 bp. The probe size in one embodiment is 27 bp, which provides a valid and efficient melting profile for HRMA.

In accordance with a method of an embodiment the present invention, an amplicon is provided which contains the locus of interest. The amplicon can be produced by any method that results in the amplification of a target nucleic acid. Amplification reactions are well known in the art, and the skilled artisan can readily use any suitable amplification reaction. PCR is perhaps the most well-known of a number of the different amplification techniques. In one embodiment, asymmetric PCR is utilized to generate the amplicon. As is well known in the art, asymmetric PCR uses one primer in a limiting concentration and the other primer in excess concentration to preferentially amplify one DNA strand in a double-stranded DNA template. It is typically used in sequencing and hybridization probing where amplification of only one of the two complementary strands is required. PCR is carried out as usual, but with an excess of the primer for the strand targeted for amplification. Because of the slow (arithmetic) amplification later in the reaction after the limiting primer has been used up, extra cycles of PCR are required. See Innis et al. (Proc Natl Acad Sci USA 85:9436-9440, 1988). A recent modification on this process, known as Linear-After-The-Exponential-PCR (LATE-PCR), uses a limiting primer with a higher Tm rather than the excess primer to maintain reaction efficiency as the limiting primer concentration decreases mid-reaction. See Pierce and Wangh (Methods Mol Med 132:65-85, 2007). In a preferred embodiment, basic asymmetric PCR is utilized to generate an amplicon having the locus of interest.

Thus, in one embodiment of a method in accordance with the present invention, asymmetric PCR is used to preferentially amplify one DNA strand of a target double stranded DNA template in order to produce amplicons having the locus of interest in order to distinguish between the locus of interest on the gene target of interest and other highly homologous regions of genomic DNA. In accordance with the present invention, the primers for asymmetric PCR are designed in order to minimize the size of the amplicon and to enhance the probe:amplicon size ratio. Also in accordance with the present invention, an unlabeled probe is utilized to distinguish between wild-type and variant DNA in the locus of interest. Design considerations for the primers and probe are described in further detail herein.

According to one aspect of the present invention, the method includes hybridizing a portion of the amplicon produced by asymmetric PCR with an unlabeled probe. A portion of the amplicon hybridizes with the unlabeled probe to produce a probe-amplicon element. The probe-amplicon element can be formed in the presence of an indicator dye, such as any of those described herein and well known to the skilled artisan. The use of the unlabeled probe described herein produces unique melting signatures. Melting signature curves can be obtained by subjecting the dyed unlabeled probe-amplicon element to high resolution thermal melting analysis as described herein. The method according to an embodiment of the present invention, e.g., the use of specially designed primers and probe, is capable of distinguishing a locus of interest from other highly homologous regions of genomic DNA and of detecting the presence of a variant in the locus of interest.

In one embodiment, a biological sample containing a target nucleic acid having a locus of interest is subjected to asymmetric PCR to generate an amplicon having the locus of interest. The asymmetric PCR can be performed in any suitable instrument for performing PCR, including thermal cyclers and microfluidic devices well known to the skilled artisan. A reaction mixture containing the small amplicon having a locus of interest is involved in hybridization with the unlabeled probe so that an amplicon-probe hybridization reaction occurs to produce a probe-amplicon element. In one embodiment, the unlabeled probe is perfectly complementary to the wild-type sequence of the target nucleic acid.

In another aspect, a system for distinguishing between a locus of interest on a target nucleic acid and other highly homologous regions of genomic DNA and identifying a variant in the locus of interest according to the present invention is provided which may comprise (a) a microfluidic device comprising a plurality of sample loading zones, each of the sample loading zones being configured to house a separate asymmetric PCR using a nucleic acid having a locus of interest, wherein the microfluidic device comprises at least one sample loading zone that is loaded with a limiting primer, an excess primer, and an unlabeled probe; (b) a HRMA device, comprising a heating element, a fluorescence excitation light source and a fluorescence collection device, configured to thermally melt probe-amplicon elements obtained from asymmetric PCRs in the sample loading zones, and to generate fluorescence derivative melting curves for the probe-amplicon elements; and (c) a fluorescence derivative melting curve analysis device configured to analyze the melting curves generated by the HRMA device so as to identify a variant in the locus of interest, which because of the design of the primers has only allowed amplification of the locus of interest in distinction to other highly homologous regions of genomic DNA. Examples of microfluidic devices that can be used according to the system of the present invention are described in Hasson et al. (U.S. Patent Application Publication No. 2010/0191482) and Knight et al. (U.S. Patent Application Publication No. 2007/0231799), as well as others disclosed herein, which are incorporated herein by reference in their entirety. Examples of possible HRMA devices configured for fluorescence measurement are illustrated in U.S. Patent Application Publication No. 2008/0003593 and Knight et al. (U.S. Patent Application Publication No. 2012/0058571) and U.S. Pat. Nos. 8,306,294 and 7,629,124, as well as others disclosed herein, which are incorporated herein by reference in their entirety. In one exemplary embodiment, a fluorescence derivative melting curve analysis device can be an appropriately programmed computer that can analyze the melting curves generated by the HRMA device as disclosed herein. Conventional high resolution thermal melt software well known to the skilled artisan, including Genotype Determinator, Melt Wizard, and Melt Viewer, can be used to recognize and compare the probe melting signatures of each genotype for targeted amplicons.

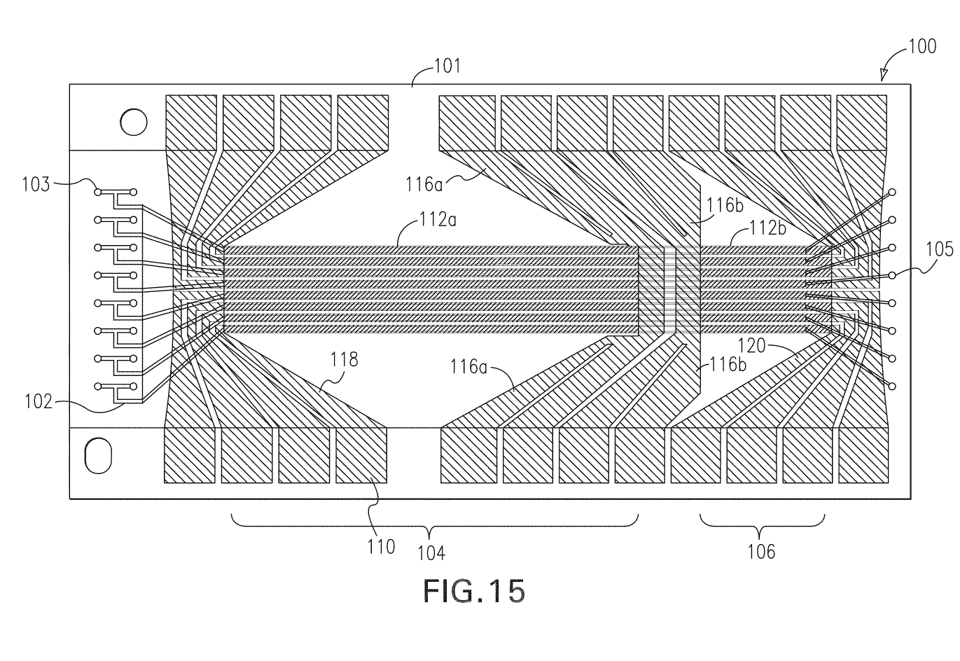

An exemplary embodiment of a system that can be used is illustrated in FIGS. 15-17. FIG. 15 illustrates a microfluidic device 100 embodying aspects of the present invention. In some embodiments, the microfluidic device 100 may be a reaction chip. In the illustrated embodiment, the microfluidic device 100 includes several microfluidic channels 102 extending across a substrate 101. Each channel 102 includes one or more inlet ports 103 (the illustrated embodiment shows two inlet ports 103 per channel 102) and one or more outlet ports 105 (the illustrated embodiment shows one outlet port 105 per channel 102). In exemplary embodiments, each channel may be subdivided into a first portion extending through a PCR thermal zone 104 and a second portion extending through a thermal melt zone 106.

In an embodiment, the microfluidic device 100 further includes thermal control elements in the form of thin film resistive heaters 112 associated with the microfluidic channels 102. In one non-limiting embodiment, the thin film resistive heaters 112 may be platinum resistive heaters whose resistances are measured in order to control their respective temperatures. In the embodiment illustrated in FIG. 15, each heater element 112 comprises two heater sections: a PCR heater 112a section in the PCR zone 104, and a thermal melt heater section 112b in the thermal melt zone 106.

In one embodiment, the microfluidic device 100 includes a plurality of heater electrodes 110 connected to the various thin-film heaters 112a and 112b. In non-limiting embodiments, heater electrodes 110 may include PCR section leads 118, one or more PCR section common lead 116a, thermal melt section leads 120, and one or more thermal melt section common lead 116b. According to one embodiment of the present invention, a separate PCR section lead 118 is connected to each of the thin-film PCR heaters 112a, and a separate thermal melt section common lead 116b is connected to each of the thin-film thermal melt heaters 112b.

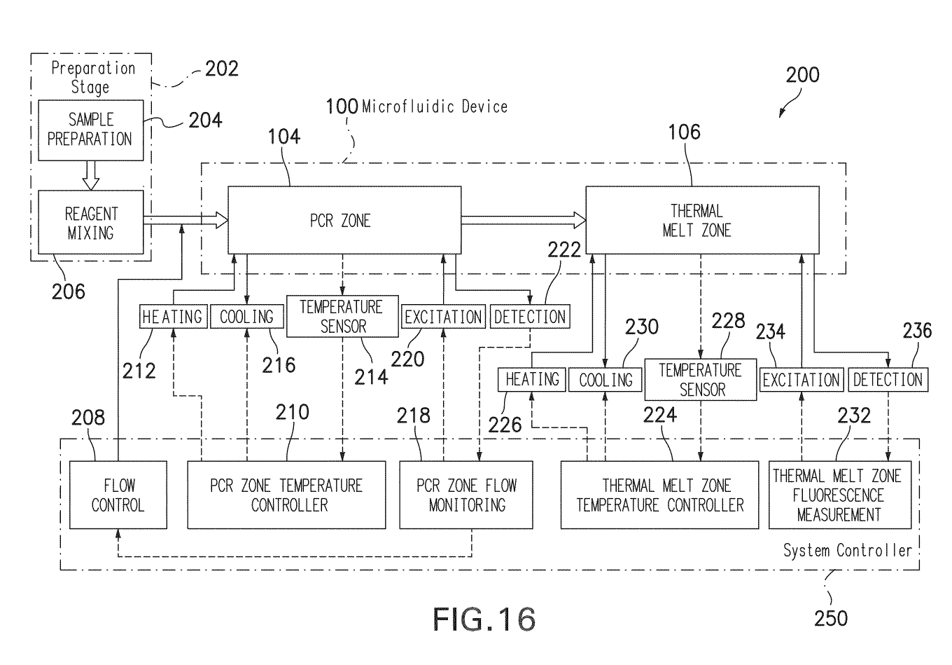

FIG. 16 illustrates a functional block diagram of a system 200 for using a microfluidic device 100, in accordance with one embodiment. The DNA sample is input in the microfluidic chip 100 from a preparation stage 202. As described herein, the preparation stage 202 may also be referred to interchangeably as the pipettor system. The preparation stage 202 may comprise appropriate devices for preparing the sample 204 and for adding one or more reagents 206 to the sample. Once the sample is input into the microfluidic chip 100, e.g., at an input port 103, the sample flows through a channel 102 into the PCR zone 104 where PCR takes place. That is, as the sample flows within a channel 102 through the PCR zone 104, the sample is exposed to the PCR temperature cycle a plurality of times to effect PCR amplification. Next, the sample flows into the thermal melt zone 106 where a high resolution thermal melt process occurs. Flow of sample into the microfluidic chip 100 can be controlled by a flow controller 208. The flow controller may be part of a control system 250 of the system 200. The control system 250 may comprise the flow controller 208, a PCR zone temperature controller 210, a PCR zone flow monitor 218, a thermal melt zone temperature controller 224, and/or a thermal melt zone fluorescence measurement system 232. In some embodiments, the control system 250 may also comprise a thermal melt zone flow monitor and/or PCR zone fluorescence measurement system. Accordingly, in some embodiments, flow control in the thermal melt zone may occur via melt zone flow monitoring. Also, the flow controller 208 may comprise a single unit that simultaneously or alternately controls flow in both the PCR and thermal melt zones, or the flow controller 208 may comprise a PCR zone flow controller and a separate thermal melt zone flow controller that independently control flow in the PCR and thermal melt zones.

The temperature in the PCR zone 104 can be controlled by the PCR zone temperature controller 210. The PCR zone temperature controller 210, which may be a programmed computer or other microprocessor or analog temperature controller, sends signals to the heater device 212 (e.g., a PCR heater 112a) based on the temperature determined by a temperature sensor 214 (such as, for example, an RTD or thin-film thermistor, or a thin-film thermocouple thermometer). In this way, the temperature of the PCR zone 104 can be maintained at the desired level or cycled through a defined sequence. According to some embodiments of the present invention, the PCR zone 104 may also be cooled by a cooling device 216 (for example, to quickly bring the channel temperature from 95° C. down to 55° C.), which may also be controlled by the PCR zone temperature controller 210. In one embodiment, the cooling device 216 could be a peltier device, heat sink or forced convection air cooled device, for example.

The flow of sample through the microfluidic channels 102 can be measured by a PCR zone flow monitoring system 218. In one embodiment, the flow monitoring system can be a fluorescent dye imaging and tracking system illustrated in U.S. Pat. No. 7,629,124, which is incorporated herein by reference in its entirety. According to one embodiment of the present invention, the channels in the PCR zone can be excited by an excitation device 220 and light fluoresced from the sample can be detected by a detection device 222. An example of one possible excitation device and detection device forming part of an imaging system is illustrated in U.S. Patent Application Publication No. 2008/0003593 and U.S. Pat. No. 7,629,124, which are incorporated herein by reference in their entirety.

The thermal melt zone temperature controller 224, e.g. a programmed computer or other microprocessor or analog temperature controller, can be used to control the temperature of the thermal melt zone 106. As with the PCR zone temperature controller 210, the thermal melt zone temperature controller 224 sends signals to the heating component 226 (e.g., a thermal melt heater 112b) based on the temperature measured by a temperature sensor 228 which can be, for example, an RTD, thin-film thermistor or thin-film thermocouple. Additionally, the thermal melt zone 106 may be independently cooled by cooling device 230. The fluorescent signature of the sample can be measured by the thermal melt zone fluorescence measurement system 232. The fluorescence measurement system 232 excites the sample with an excitation device 234, and the fluorescence of the sample can be detected by a detection device 236. An example of one possible fluorescence measurement system is illustrated in U.S. Patent Application Publication No. 2008/0003593 and U.S. Pat. No. 7,629,124, which are incorporated herein by reference in their entirety.

In accordance with aspects of the present invention, the thin film heaters 112 may function as both heaters and temperature detectors. Thus, in one embodiment of the present invention, the functionality of heating element 212 and 226 and temperature sensors 214 and 228 can be accomplished by the thin film heaters 112.

In one embodiment, the system 200 sends power to the thin-film heaters 112a and/or 112b, thereby causing them to heat up, based on a control signal sent by the PCR zone temperature controller 210 or the thermal melt zone temperature controller 224. The control signal can be, for example, a pulse width modulation (PWM) control signal. An advantage of using a PWM signal to control the heaters 212 is that with a PWM control signal, the same voltage potential across the heaters may be used for all of the various temperatures required. In another embodiment, the control signal could utilize amplitude modulation or alternating current. It may be advantageous to use a control signal that is amplitude modulated to control the heaters 212 because a continuous modest change in voltage, rather than large voltage steps, avoids slew rate limits and improves settling time. Further discussion of amplitude modulation can be found in U.S. Patent Application Publication No. 2011/0048547, which is incorporated herein by reference in its entirety. In another embodiment, the control signal could deliver a steady state power based on the desired temperature. In some embodiments, the desired temperature for the heaters is reached by changing the duty cycle of the control signal. For example, in one non-limiting embodiment, the duty cycle of the control signal for achieving 95° C. in a PCR heater might be about 50%, the duty cycle of the control signal for achieving 72° C. in a PCR heater might be about 25%, and the duty cycle of the control signal for achieving 55° C. in a PCR heater might be about 10%.

The microfluidic device 100 and the system 200 can be used in conjunction with aspects of the present invention. For example, one can obtain multiple reagents, mix them, deliver them to a microfluidic device (e.g., an interface chip), and utilize the flow controller 208 to create fluid segments that flow through the microfluidic device 100 with minimal mixing between the fluid segments, in accordance with aspects of the invention.

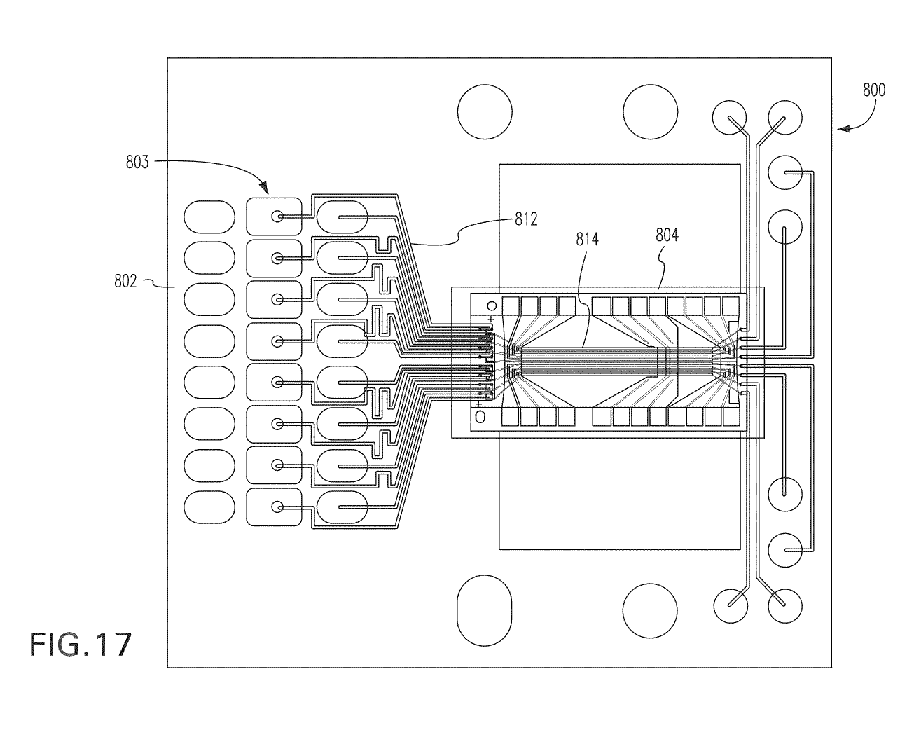

FIG. 17 illustrates a microfluidic chip system 800 for providing fluid segments that move through a microfluidic chip with minimal mixing between serial segments, in accordance with some embodiments of the present invention. In the non-limiting exemplary embodiment of FIG. 17, the microfluidic chip system 800 includes an interface chip 802 and a reaction chip 804. In some embodiments, the interface chip 802 can contain access tubes (e.g., capillary tubes or other tubes) or wells 803 that allow different reaction mixtures (i.e., fluids) to be entered into the microfluidic system in series, such as by the process 600 described above. In some embodiments, the reaction chip 804 is a smaller chip that carries out the reaction chemistry, such as PCR and thermal melting. In some embodiments, the reaction chip 804 may be a microfluidic device such as the microfluidic device 100.

In one embodiment, amplification of the biological sample having a locus of interest and annealing and extension of the amplicons produced by amplification are performed in a microfluidic device. In one embodiment, the microfluidic device has a plurality of channels for amplification, annealing and extension of one or more biological samples, or one or more portions of one biological sample, in parallel. In one embodiment, the microfluidic device has a plurality of wells for loading the biological sample onto the device. In another embodiment, the microfluidic device has a first part for performing PCR amplification and a second part for performing thermal melt analysis. A description of PCR amplification, and examples of microfluidic devices including thermal control elements for PCR amplification and thermal melt analysis are provided in U.S. Patent Application Publication Nos. 2009/0248349, 2009/0318306, 2010/0233687 and 2011/0056926 and U.S. Pat. No. 8,306,294, the entire disclosures of which are incorporated herein by reference.

Embodiments of the present invention can be used in a variety of instruments but are particularly useful in PCR and thermal melt systems that perform in vitro diagnostics. Embodiments of the present invention may be used in devices that are intended for thermal melt of samples (diagnostics) as well as other heaters and sensors within the instrument that perform entirely different functions (e.g., sample prep or PCR). Embodiments of the present invention may be used in both microfluidic and non-microfluidic devices. Examples of microfluidic devices known in the art include, but are not limited to, Chow et al. (U.S. Pat. No. 6,447,661), Kopf-Sill (U.S. Pat. No. 6,524,830), Spaid (U.S. Pat. No. 7,101,467), Dubrow et al. (U.S. Pat. No. 7,303,727), Schembri (U.S. Pat. No. 7,390,457), Schembri (U.S. Pat. No. 7,402,279), Takahashi et al. (U.S. Pat. No. 7,604,938), Knapp et al. (U.S. Patent Application Publication No. 2005/0042639), Hasson et al. (U.S. Patent Application Publication No. 2010/0191482) and Knight et al. (U.S. Patent Application Publication No. 2012/0058571), as well as others disclosed herein. Each of these patents or patent application publications is incorporated herein by reference in its entirety.

In one or more embodiments of the invention, after amplification, annealing and extension, the probe-amplicon elements are subjected to saturation dyes and high resolution melting analysis in order to generate melting curves. When the targeted sequence contains disease or disorder-causing mutations/variants, during melting, the melting signatures of these mutations/variants can be definitively displayed on the probe melting region on the melting curve. Accordingly, analysis of the generated melting curves using the unlabeled probe for the locus of interest on target nucleic acid makes it possible to discern between wild-type and variant alleles. An example of one possible fluorescence measurement system is illustrated in U.S. Patent Application Publication Nos. 2008/0003593 and U.S. Pat. Nos. 8,306,294 and 7,629,124, which are incorporated herein by reference in their entirety.

In some embodiments, the amplicon having a locus of interest is produced by mixing a target nucleic acid having a locus of interest with a first primer and a second primer, where the primers are designed to amplify the target nucleic acid having a locus of interest and to distinguish the locus of interest from highly homologous genomic regions, and amplifying the target nucleic acid having a locus of interest to generate an amplicon having the locus of interest. In other embodiments, the amplicon having a locus of interest is produced using asymmetric PCR which utilizes an excess primer and a limiting primer.

In one embodiment, the excess primer hybridizes to a unique heterologous region in the locus of interest as compared to the other highly homologous genomic regions. In another embodiment, the excess primer is a forward primer. In one embodiment, the limiting primer hybridizes as close as possible to the variant in the locus of interest to minimize amplicon size. In another embodiment, the limiting primer is a reverse primer. In one embodiment, the probe hybridizes to the wild-type sequence. In another embodiment, the probe hybridizes to the variant sequence. In an additional embodiment, the probe can be unlabeled. In an additional embodiment, the melting curve is generated once the limiting primer is exhausted.

In one embodiment, the locus of interest is highly homologous to one or more genomic regions and the method distinguishes the locus of interest from the other highly homologous genomic regions.

In another aspect, the present invention also provides a method of designing primers and probes that are useful for thermal melt analysis of a locus of interest that contains a disease or disorder-causing variant on a target nucleic acid that is highly homologous to other genomic regions. In accordance with this aspect, the method may comprise (a) selecting a locus of interest of a disease, in which the locus of interest has a disease or disorder-causing variant and in which the locus of interest is on a nucleic acid that is highly homologous with other genomic regions, (b) designing a pair of primers for use in asymmetric PCR, and (c) designing a probe for hybridizing to one strand of the locus of interest. In one embodiment, one primer is designed to hybridize to a unique heterologous region in the locus of interest as compared to the other highly homologous genomic regions. In another embodiment, this primer is the excess primer. In an additional embodiment, the excess primer is a forward primer. In one embodiment, one primer is designed to hybridize as close as possible to the variant in the locus of interest to minimize amplicon size. In another embodiment, this primer is the limiting primer. In an additional embodiment, the limiting primer is a reverse primer. In one embodiment, the probe hybridizes to the wild-type sequence. In another embodiment, the probe hybridizes to the variant sequence. In one exemplary embodiment, the primers and probe are designed using an appropriately programmed computer.

In some embodiments, a primer pair is designed so that a forward primer is set as the excess primer and a reverse primer is set as the limiting primer in the asymmetric PCR. In other embodiments, a primer pair is designed so that a forward primer is set as the limiting primer and a reverse primer is set as the excess primer in the asymmetric PCR. The excess primer is designed to anneal to a unique region in the locus of interest so that it can be distinguished from highly homologous genomic regions. Each primer is designed so that the amplicon size can be minimized for high genotyping sensitivity. The unique region of the locus of interest can be determined using conventional techniques, such as BLAST analysis and USCS Human BLAT SEARCH GENOME analysis.

Once a unique region is identified, one primer is designed to anneal to this region and the second primer is designed to anneal in the homologous region (i.e., the region that is homologous between the locus of interest and the other highly homologous genomic regions) using conventional techniques. For example, primer design can then be implemented, e.g., by an appropriately programmed computer. Examples of primer design software include, but are not limited to, Primer 3 software (frodo.wi.mit.edu/primer3/) or SNP Wizard software (courtesy of Carl Wittwer, University of Utah) to produce primer sequences. Sequence similarity of primers output by the software and other sequences within the same genome are compared using tools such as BLAST and Human BLAST to obtain sequence alignments. If the primer sequences are the same as other sequences within the same genome on multiple locations, e.g. on different chromosomes, or on the same chromosome, this primer is rejected. If the primer sequence is a unique sequence, which, in a preferred embodiment, is 100% different from any other sequences, this primer and/or probe are accepted. Once primer pairs are designed as described above and accepted, the primer pairs are checked using an appropriately programmed computer with, for example, In-Silico PCR tool for PCR prediction for PCR conditions and products. An example of an In-Silico PCR tool includes, but is not limited to, (genome.ucsc.edu/cgi-bin/hgPcr?wp_target=&db=hg18&org=Human&wp_f=&wp_r=&wp_size=4000&wp_perfect=1 5&wp_good=15&wp_showPage=true&hgsid=151501082).

The accepted sequences can be examined for theoretical melting prediction using an appropriately programmed computer. Examples of melt prediction software include, but are not limited to, UMelt software (courtesy of Carl Wittwer, University of Utah, www.dna.utah.edu/umelt/um.php) and Poland Melt Prediction software (www.biophys.uni-duesseldorf.de/local/POLAND/). In some embodiments, the wild type sequence and the mismatched sequence of the targeted mutation can be checked with two or more programs. The predicted results can be compared, recorded, and later used for comparing with experimental data as references. Once the primer sequences have passed the above criteria and checks, they can be tested for the assay feasibility, including sensitivity, specificity, and reproducibility of the assay.

In certain embodiments, an unlabeled probe is designed that has a sequence that is complementary to the wild-type sequence. In one embodiment, the probe is designed under the same criteria as the primers for identical PCR conditions. After identifying the mutation and the locus of interest on a gene, probe design can be performed by placing the probe within the locus of interest region. The sequence can be aligned using the BLAST or HumanBlast tools previously mentioned. Using these tools, any probe sequences that are homologous to other regions of unrelated sequences should be excluded. In some embodiments, the probe is designed so as to have its Tm be less than about 70° C. In certain embodiments, the probe is designed so as to have its Tm be less than the Tm of the primers. In some embodiments, the probe is designed so that the targeted nucleic acid variant is located at approximately the middle of the length of the probe. In other embodiments, the probe is designed so that the variant is closer to the 5′ end of the probe. In further embodiments, the probe is designed so that the variant is closer to the 3′ end of the probe.

In some embodiments, the unlabeled probe is perfectly complimentary to the wild-type sequence on the forward strand of the target nucleic acid. In other embodiments, the unlabeled probe is perfectly complimentary to the wild-type sequence on the reverse strand of the target nucleic acid. Thus, in individuals having a mutation/variant at the locus of interest, the unlabeled probe will have one or more single base pair mismatch(es) at the corresponding locus. This design is used to increase the assay sensitivity and specificity of the mutation/variant at the locus of interest on the target nucleic acid. Thus, in one embodiment, the length of the unlabeled probe is designed so as to have sufficient sensitivity and specificity to the mutation/variant within the locus of interest. The length of the unlabeled probe can be varied depending on the melting characteristics of the targeted amplicon.

The probe size is maximized in order to maximize the probe:amplicon ratio. In some embodiments, the probe is between 20 and 50 nucleotides, such as between 25 and 40 nucleotides, such as between 25 and 30 nucleotides. In some embodiments, the probe is blocked at the 3′ end to prevent extension of the probe. The 3′ end may be blocked with any suitable blocker, for example, a 3′ C6-amino blocker, a 3′ phosphorylation blocker, or any other suitable 3′ blockers. During probe design, the range of optimal Tms of probe is about 2-10° C. lower than primer Tms. In one embodiment, the probe Tm is less than about 4-8° C. lower than primer Tms. In another embodiment, the probe Tm is about 4° C. lower than lowest primer Tm.

According to one or more of the above embodiments, the present invention provides a method for detecting a disease in a patient based on the patient's genotype in a locus of interest that is distinguished from other highly homologous regions of genomic DNA as a result of the primer design described herein. In one embodiment, diagnosticians can take advantage of a priori knowledge of disease or disorder-causing variant gene sequences associated with a target disease or disorder. In another embodiment, it is possible to use the present invention as a tool to identify and genotype disease or disorder-causing variants associated with a particular disease or disorder based on analysis of the target sequence and melting signature curves obtained by using the unlabeled probe. In one embodiment, a portion of a biological sample from a patient can be subjected to asymmetric PCR to produce an amplicon having a locus of interest, which hybridizes to an unlabeled probe to produce a melting curve.

In another embodiment, a biological sample from a patient can be subjected to asymmetric PCR to produce an amplicon having a locus of interest. A portion of the amplicon having a locus of interest is subjected to a unlabeled probe assay to produce a melting curve.

The present invention provides a method to distinguish a locus of interest that may carry a variant from other highly homologous genomic regions. This distinction is made on the basis of the selection of the primers as described herein, such that the asymmetric PCR amplification results in the production of an amplicon of the locus of interest and no amplification of other highly homologous genomic regions.

As described in one embodiment herein, an unlabeled probe assay includes the steps of hybridizing an unlabeled probe to a locus of interest on an amplicon to form a probe-amplicon element, adding a saturated dye to the probe-amplicon element to form a mixture, and generating a melting curve for the probe-amplicon element by measuring fluorescence from the dye as the mixture is heated.

The above described embodiments of an unlabeled probe assay in high resolution thermal melt analysis is simple, fast and can be easily adapted to both microfluidic and non-microfluidic devices. The probe melting signatures of each genotype for targeted amplicons can be recognized. In one embodiment, the recognition can be made using an appropriately programmed computer. In an exemplary embodiment, conventional high resolution thermal melt software well known to the skilled artisan, including Genotype Determinator and Melt Viewer, can be used. The definitive probe melting shapes of each genotype per each mutation generated with the probe can be used in a clinical molecular diagnostic report. The definitive probe melting shapes of each genotype per each mutation generated with the probe can be accepted and interpreted easily and clearly by clinicians and physicians. Probe/primer sets designed according to the present invention can be used to study various diseases, including, for example, Cystic Fibrosis.

In another aspect, the present invention provides a kit for distinguishing between a locus of interest and other highly homologous regions of genomic DNA and detecting a disease or disorder in a patient based on the patient's genotype. An unlabeled probe designed according to the present invention, along with primer pairs designed according to the present invention, can be included in a kit for performing a diagnostic test for detecting a specified disease or disorder for which the probes and primers were designed. The kit may also be used for conducting biochemical studies on various nucleic acid sequences. The kit may include instructions for performing a diagnostic test. In a non-limiting example, a kit can contain primer pairs, an unlabeled probe, as well as instructions for performing a diagnostic test for detecting variants/mutations using a target nucleic acid having a locus of interest. The kit can also contain common reagents necessary for PCR such as polymerases, ligases, NADP, dNTPs, buffers, salts, etc. Such reagents are known to persons of ordinary skill in the art. In one embodiment, the kit can include a primer having a nucleotide sequence set forth in SEQ ID NO:5, a primer having a nucleotide sequence set forth in SEQ ID NO:6 and a probe having a nucleotide sequence set forth in SEQ ID NO:7. In another embodiment, the instructions may include instructions for performing the diagnostic test using the methods and systems described herein.

Unlabeled probe and primer pairs designed according to the present invention can be used in any high resolution thermal melt analysis instrument for clinical molecular diagnosis on a target disease or disorder. Unlabeled probe and primer pairs designed according to the present invention can also be used by clinic laboratories for clinical molecular diagnosis of a disease or disorder such as, for example, Cystic Fibrosis. Unlabeled probe and primer pairs designed according to the present invention can be used as a reflexive genotyping test following HRMA scanning of a gene by clinic laboratories for the clinical molecular diagnosis of a target disease.

The design concepts of the present invention can be applied to situations on other genes of interest as well as to mutation discovery for other genes which have highly homologous genomic regions.

As illustrated herein, the primers described in one embodiment of the present invention allow the amplification of the exon 9 region of the CFTR gene without co-amplification of the highly homologous region on chromosome 20. The unlabeled probe described in one embodiment of the present invention can be used to genotype DNAs for the A455E mutation using HRMA of the unlabeled probe melt region. This unlabeled probe approach for genotyping the exon 9 A455E mutation described herein in one embodiment of the present invention is accurate and simple. The assay is a useful diagnostic assay with quick turn-around time. In addition to the A455E variant, the primer set described in one embodiment of the present invention can also be used to scan for the exon 9 1461ins4 variant. The ACOG recommended screening panel for CF includes the A455E mutation. The primer/probe design described in one embodiment of the present invention can correctly genotype the A455E mutation.

### EXAMPLES

The present invention is described by reference to the following Examples, which are offered by way of illustration and are not intended to limit the invention in any manner. Standard techniques well known in the art or the techniques specifically described below were utilized.

### Example 1

The present invention takes into consideration the design of primers to differentiate exon 9 of the CFTR gene from other homologous sequences on the human genome. The advantages of using an unlabeled probe method for detecting the mutation of Exon 9 A455E can be essential. The method according to an embodiment of the present invention differentiates exon 9 of the CFTR gene from other homologous regions on the human genome by locating the forward primer in the heterologous region on intron 8 junction region and to identify the mutation of A455E using an unlabeled probe. This embodiment of the present invention provides a convincing result for the A455E detection, as well as avoiding the pesudogene errors.

The present invention provides a unique and convincing methodology for mutation identification, including as to a sequence that includes A455E on exon 9 and its junction intron 8 region that differentiate exon 9 from the homologous regions, which is performed using an unlabeled probe approach to characterize the A455E mutation with HRMA. The present invention can be applied to HRMA systems as described above and as otherwise known in the art.

The unlabeled probe approach for genotyping A455E on exon 9 is an accurate and simple method, and will fit the art-recognized need for a quick turn-around test.

The primer and probe design according to an embodiment of the present invention provides reliable results for the clinical diagnosis of the A455E variant in the CFTR gene.

Specifically, in this approach, the forward primer anneals in the unique region in intron 8 as indicated in FIG. 6. The reverse primer anneals just downstream of the A455E mutation in order to minimize the amplicon size. An unlabeled probe was also designed to expressly target the A455E mutation. The purpose of this design was to specifically amplify only the CFTR exon 9 without also amplifying the pseudogene regions on other chromosomes. Normally, both PCR primers are designed to anneal to unique genomic sequence to prevent unwanted secondary product formation. Due to the existence of pseudogene regions on other chromosomes, as well as the need to minimize amplicon size, the normal primer design strategy was not possible. Table 1 summarizes the final oligonucleotide designs.

TABLE 1
Oligonucleotide Design

| | | | | GC |
|---|---|---|---|---|
| Oligo | | Length | Tm | Content |
| Name | Sequence (5′-3″) (SEQ ID NO:) | (bp) | (° C.) | (%) |
| Exon 9 | gggccatgtgcttttcaaact (5) | 21 | 64 | 48 |
| A455E F1 | | | | |
| Exon 9 | gaactacCTTGCCTGCTCCA (6) | 20 | 60 | 55 |
| A455E R1 | | | | |
| E9 A455E | AACCGCCAACAACTGTCCTCTTTCTAT (7) | 27 | 56 | 32 |
| UP 1r | | | | |

The amplicon generated from the two primers is 304 bp. The probe size was made as large as possible while maintaining an adequate GC content in order to maximize the probe:amplicon size ratio necessary for a good probe signal during high resolution melt analysis.

Thus, in accordance with an embodiment of the present invention, the following principles guided the oligonucleotide and assay design:

1) The forward primer, exon 9 A455E F1, was designed in the unique region of intron 8 to differentiate the exon 9 region from other homologous genomic regions.

2) The reverse primer, exon 9 A455E R1, was designed as close as possible to the A455E mutation to minimize the amplicon size.

3) The unlabeled probe, e9 A455E UP1r, was designed to be complementary to the wild-type sequence. In individuals with the A455E mutation, the probe has a one base pair mismatch at the A455E locus. Ideally, the mismatch would be designed in the center of the probe. This was not possible in this case, as the probe anneals close to the end of the amplicon. The probe mismatch was therefore designed to be closer to the 5′ end of the probe, as previous research has suggested that in the less ideal cases, where centered mismatches are not possible, unlabeled probe genotyping is more successful when the mismatched base-pair is closer to the 5′ end of the probe (Unlabeled Probe Project, Rob Pryor, University of Utah).

4) The probe size was maximized in order to maximize the probe:amplicon size ratio. The smaller the ratio, the higher the probe signal will be during high resolution melt analysis.

5) The probe was blocked at the 3′ end with an amino-C6 modification in order to prevent extension by the DNA polymerase during PCR. This 3′ modification was used to generate the data outlined below. Other suitable 3′ modifications, such as a 3′ phosphorylation modification, can also be utilized.

6) The reverse primer was the limiting primer in the asymmetric PCR reaction. As the reverse primer anneals to the non-unique sequence in exon 9, the product produced from extension of this primer should be limited.

7) The primer sequences could also be used with the synthetic construct DNA (described further below). The primer annealing sites are within the 600 bp plasmid vector insert which allows PCR amplification not only of genomic DNA exon 9 targets but of synthetic construct DNA exon 9 targets as well.

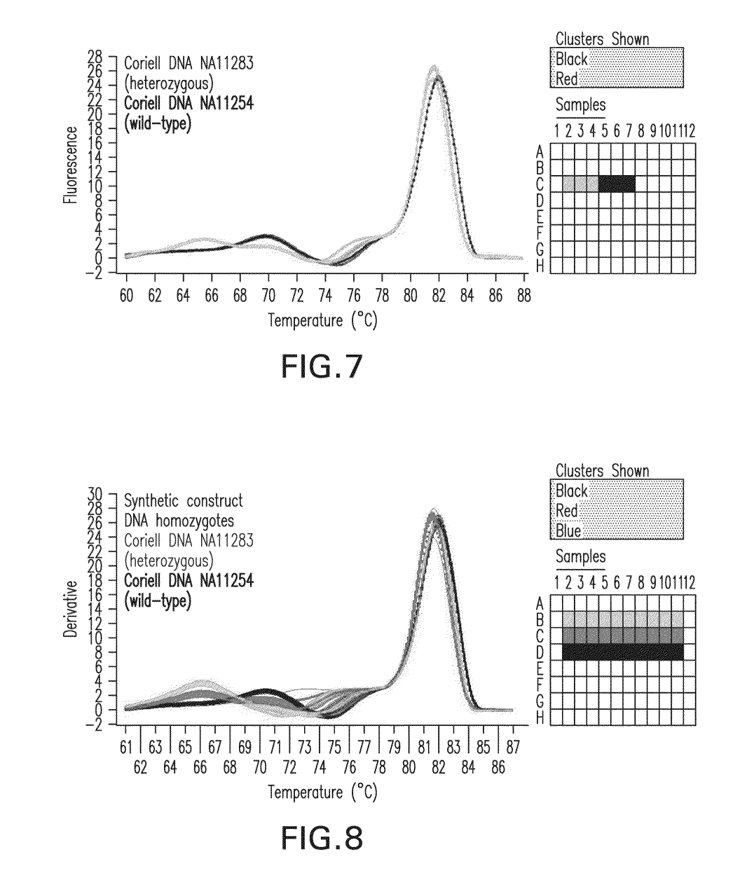

The preliminary data obtained by utilizing the new primer and probe design showed that the CFTR exon 9 A455E target was successfully genotyped by the unlabeled probe designed in in accordance with the present invention. FIG. 7 shows genotyping results with two different genomic DNAs. The wild-type DNA showed the expected single peak in the probe melt region, and the heterozygous DNA showed the expected double peak in the probe melt region.

### Example 2

In addition, synthetic DNA (described further below) was used in this assay as a further check on its accuracy. 600 bp of synthetic DNA contained the exon 9 region, and the only base-pair change contained in that design was a homozygous change from C to A. This DNA is expected to produce a single probe melt peak corresponding to the lower temperature heterozygous DNA peak. Experimental data (FIG. 8) showed that all DNAs genotype as expected. Each observed probe peak corresponds to the expected probe peak.

### Example 3

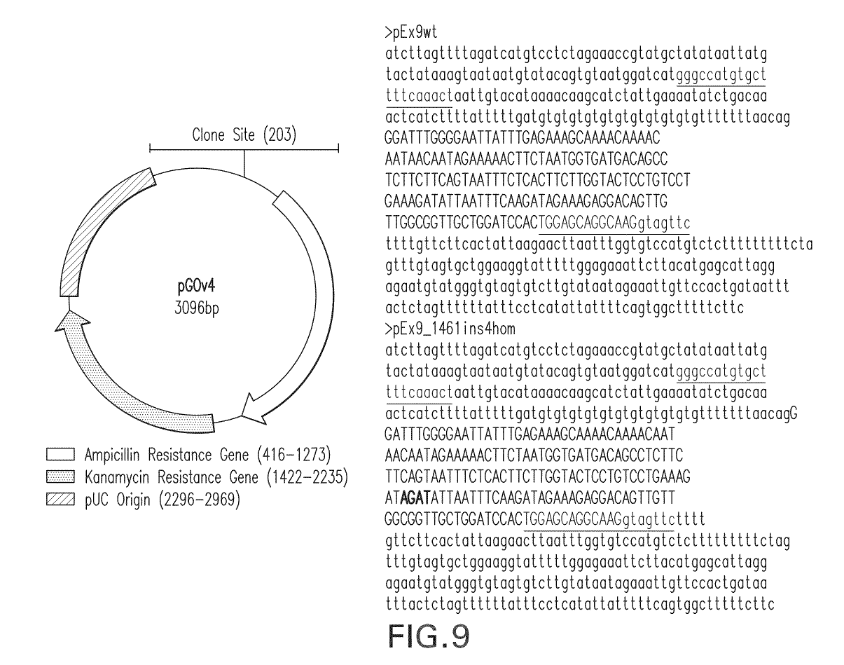

The new primer set design was further tested to scan for the exon 9 1461ins4 mutation. Genomic wild-type DNA and synthetic construct DNAs harboring the mutation were both tested. Briefly, the constructs consist of an approximately 600 bp sequence inserted into a pGOv4 plasmid vector (FIG. 9). The plasmid was replicated by the E. coli host cell, and the host cells were easily grown as needed. Plasmids were purified from the host. Plasmid inserts were designed to have either wild-type sequence or sequence containing a homozygous mutation (FIG. 9). After the plasmids were extracted and purified from the host, wild-type plasmids and homozygous plasmids were mixed together in a 1:1 ratio to produce a heterozygous construct DNA mix.

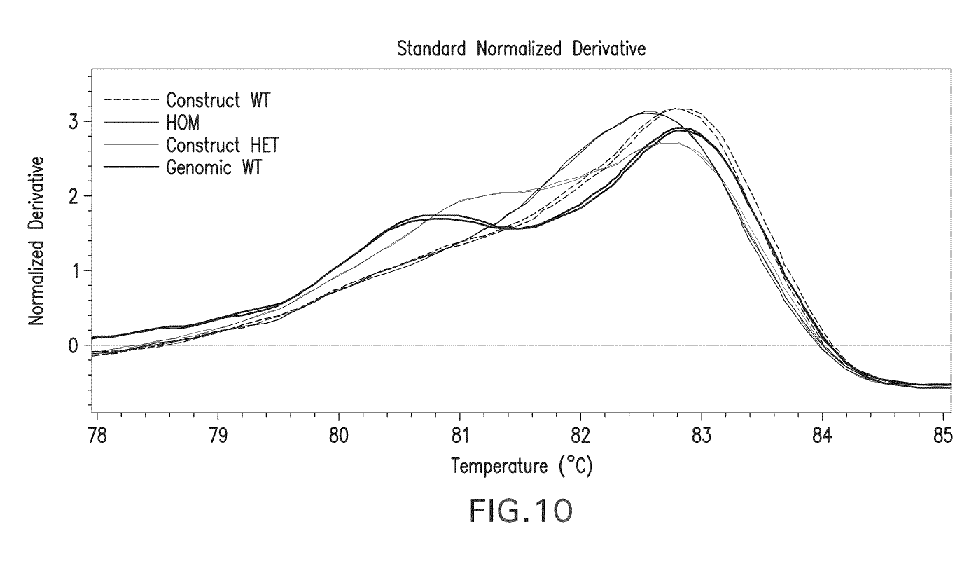

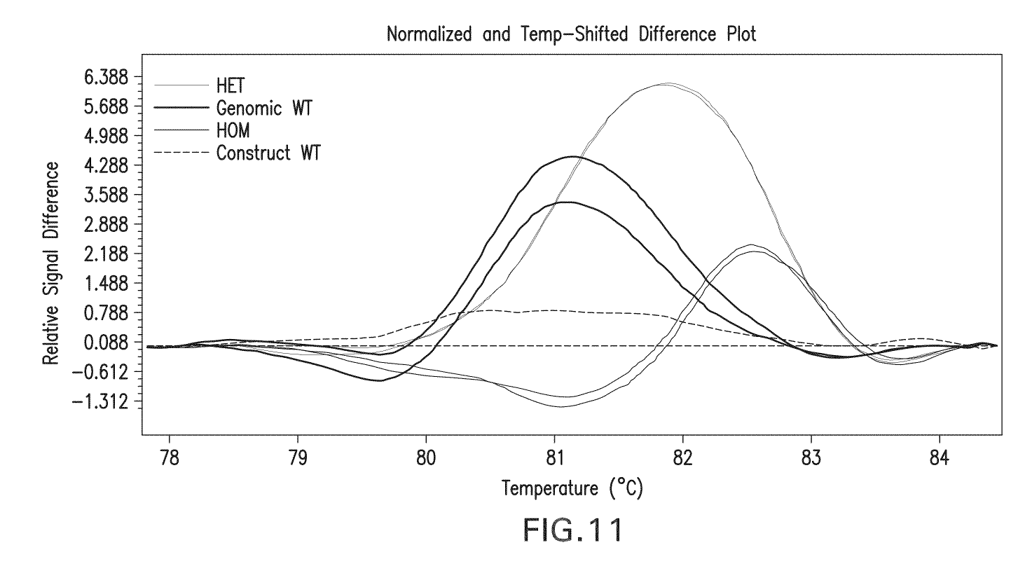

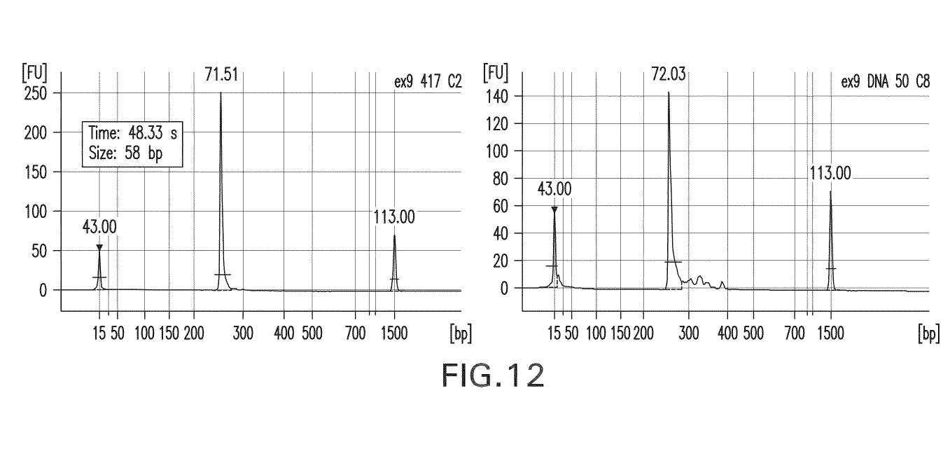

A different primer design (other than the one described above in accordance with one embodiment of the present invention) was initially used. The primers that were used have the sequences TGGGGAATTATTTGAGAAAG (SEQ ID NO:8) and CTTCCAGCACTACAAAC TAGAAA (SEQ ID NO:9), and the use of these primers resulted in an amplicon of 258 bp of exon 9 (Montgomery, J. Wittwer, C. Kent J. Zhou, L., Clin. Chem 53:11 1891-1898 (2007) supplementary material). This primer set, as is clear from FIG. 10, mistakenly amplified the exon 9 pseudogene region. Scan results (FIG. 11) further supported the existence of a melt discrepancy between the construct wild-type and the genomic wild-type DNAs. The construct DNAs do not include the entire genome, and therefore represent what a specific PCR product should look like. In contrast, the genomic wild-type DNA does include the chromosome 20 region with the CFTR exon 9 pseudogene. The two melt domains observed for the genomic DNA in FIG. 10 can be explained by the original primer set amplifying two regions on the genome: one from chromosome 7 (the true gene) and one from chromosome 20 (the pseudogene). As these two regions are homologous in sequence, both PCR products are the same size and as such were indistinguishable on Bioanalyzer analysis (FIG. 12).

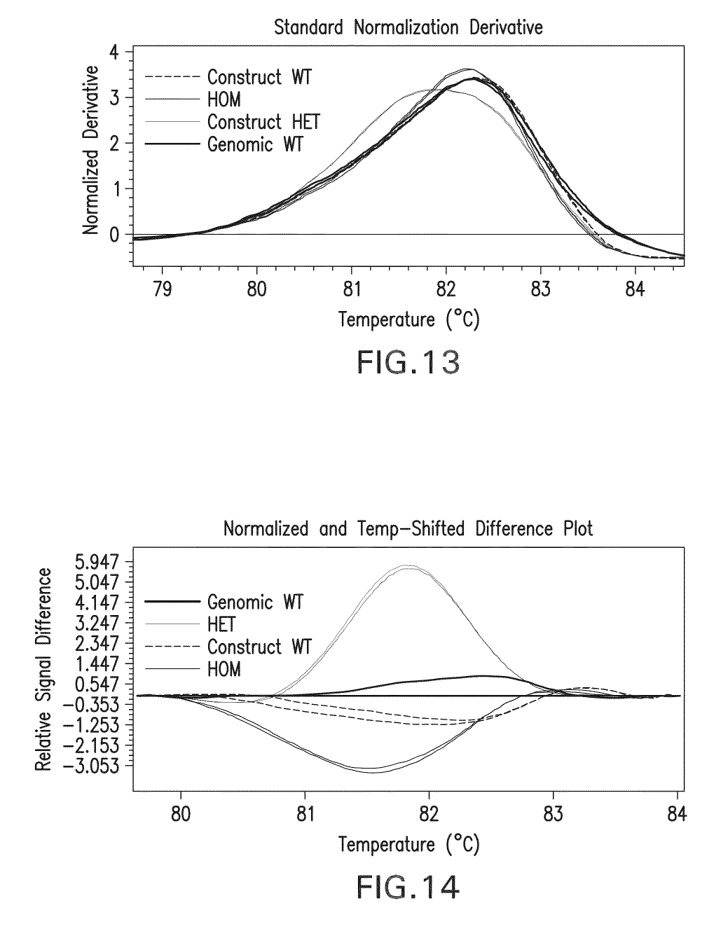

When the primer set described above in accordance with the present invention was utilized, the second melt feature observed in FIG. 10 for the genomic DNA was no longer apparent (FIG. 13). Scan results provided additional evidence that this primer set produced a specific product (FIG. 14). Moreover, the construct heterozygote mix and the construct homozygote DNA each produced a distinctive melt pattern as compared with both of the wild-type DNAs. This experiment showed the utility of the primer set in accordance with one embodiment of the present invention both for gene scanning experiments as well as its ability to be used in the CFTR synthetic construct project.

The present invention allows differential amplification of the CFTR exon 9 from other homologous sequences on the human genome during PCR. The advantage of using an unlabeled probe method to detect the exon 9 A455E mutation is that it is faster and cheaper than sequencing. A small amplicon approach which does not utilize a specific probe was not possible. Small amplicon genotyping requires, as the name implies, the generation of a small (typically 100 bp or less) PCR product. The existence of so much homologous sequence around the A455E mutation prevented specific primer design which could generate a small product. An unlabeled probe was therefore needed to pick out the A455E mutation from a larger, approximately 300 bp PCR product. The specificity of the forward primer differentiates exon 9 from other homologous regions on the human genome. This design permits accurate A455E genotyping and avoids amplification of pseudogene sequence during PCR.

### Example 4

The present invention provides a method for the design of primers and probes that can be used for distinguishing a locus of interest on a target nucleic acid from other highly homologous genomic regions and for identifying a mutation that may be present in the locus of interest.

1. The design of the unlabeled probe and the primer pair is simple and straight forward;

2. In one embodiment, if the unique region of the target locus is on the 5′ side of the mutation, the forward primer is designed to anneal to this unique region. In one embodiment described herein, the forward primer was designed to anneal to the unique region in intron 8 of the CFTR gene to amplify the desired CFTR exon 9 region. In another embodiment, if the unique region of the target locus is on the 3′ side of the mutation, reverse primer is designed to anneal to the unique region.

3. In one embodiment, if the forward primer anneals to the unique region, the reverse primer is designed to anneal as close to the mutation as possible to minimize amplicon size. In one embodiment described herein, the reverse primer was designed to anneal close to the A455E mutation in exon 9 of the CFTR gene to minimize amplicon size. In another embodiment, if the reverse primer binds to the unique region, the forward primer is designed to anneal as close to the mutation as possible to minimize amplicon size.

4. The unlabeled probe is designed to be perfectly complementary to the wild-type DNA sequence. In one embodiment described herein, individuals with the A455E mutation have a single by mismatch to the probe, which provides a different pattern during HRM analysis.

5. In one embodiment, if the forward primer binds to the unique region, the reverse primer is the limiting primer to reduce the odds that PCR amplification of the pseudogene will occur. In one embodiment described herein, the reverse primer anneals to non-unique sequence in exon 9 of the CFTR gene and therefore the product produced from extension of this primer should be limited. In another embodiment, if the reverse primer binds to the unique region, the forward primer is the limiting primer to reduce the odds that PCR amplification of the pseudogene will occur.

6. In one embodiment, if the forward primer binds to the unique region, the forward primer is added to the PCR in excess in order to generate the maximum amount of the desired specific product for the probe to anneal to for HRMA. In one embodiment described herein, the forward primer was added to the PCR in excess in order to generate the maximum amount of the specific CFTR exon 9 PCR product for the probe to anneal to for HRMA. In another embodiment, if the reverse primer binds to the unique region, the reverse primer is added to the PCR in excess in order to generate the maximum amount of the desired specific product for the probe to anneal to for HRMA.

7. The designed oligonucleotides are generic enough to be used on any platform.

8. The pattern produced by the probe during HRM is easily recognized by HRMA software, including CULS-developed GDMV, Roche Lightcycler 480 software, and Wittwer Lab's Melt Wizard.

9. As shown herein, the primers designed in accordance with the present invention can also be used to genotype synthetic construct DNAs in addition to genomic DNAs.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. In addition, recitation of ranges of values herein are merely intended to serve as a shorthand method of referring to all separate ranges falling within the range, unless otherwise indicated, and each separate range is incorporated into the specification as if it were individually recited herein. For example, if the range 10-15 is disclosed, then the ranges 10-14, 10-13, 10-12, 10-11, 11-15, 11-14, 11-13, 11-12, 12-15, 12-14, 12-13, 13-15, 13-14 and 14-15 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of identifying a variant on a target strand of a nucleic acid molecule having a locus of interest that is substantially homologous to a second genomic region, comprising:
(a) incubating an aliquot of said nucleic acid with a limiting primer, an excess primer, and a probe that is designed to hybridize to said locus of interest on a target strand of said nucleic acid;
(b) performing asymmetric PCR using said aliquot to produce an excess of amplicons corresponding to the target strand to which the probe hybridizes, thereby producing a probe-amplicon element;
(c) generating a melting curve for the probe-amplicon element in a mixture with a saturating binding dye by measuring fluorescence from the dye as the mixture is heated;
(d) analyzing the melting curve to determine whether a variant is present.

2. The method of claim 1, wherein the target strand of a nucleic acid molecule having a locus of interest is amplified and the second genomic region is not amplified.

3. The method of claim 1, wherein the probe is unlabeled.

4. The method of claim 1, wherein the probe is blocked at its 3′ end.

5. The method of claim 1, wherein the probe has a sequence that is complementary to a wild-type sequence of the gene.

6. The method of claim 1, wherein the Tm of the probe is less than about 5° C. lower than the lowest Tm of the excess primer and the limiting primer.

7. The method of claim 1, wherein the excess primer anneals to a sequence that is unique on the target strand of a nucleic acid molecule having a locus of interest and the limiting primer anneals to a sequence that is substantially homologous to the second genomic region and set close to the variant to minimize amplicon size for high genotyping sensitivity.

8. The method of claim 1, wherein the variant is A455E of the CFTR gene.

9. The method of claim 1, wherein the variant is 1461ins4 of the CFTR gene.

10. The method of claim 8, wherein the excess primer has a nucleotide sequence of 5′-gggccatgtgcttttcaaact-3′ (SEQ ID NO:5).

11. The method of claim 8, wherein the limiting primer has a nucleotide sequence of 5′-gaactaccttgcctgctcca-3′ (SEQ ID NO:6).

12. The method of claim 8, wherein the probe has a nucleotide sequence of 5′-aaccgccaacaactgtcctctttctat-3′ (SEQ ID NO:7) and is blocked at its 3′ end.

13. A method of detecting a disease or disorder in a patient based on said patient's genotype and a priori knowledge of disease or disorder-causing variant gene sequence associated with said disease or disorder, wherein the variant gene sequence is on a target strand of a nucleic acid molecule having a locus of interest that is substantially homologous to a second genomic region, the method comprising:
(a) obtaining a biological sample from a patient, wherein the sample contains a nucleic acid molecule having the locus of interest and a second genomic region substantially homologous to the locus of interest;
(b) subjecting a portion of the biological sample to asymmetric PCR involving a limiting primer, an excess primer, and a probe to produce a probe-amplicon element;
(c) generating a melting curve by subjecting said the probe-amplicon melting element to high resolution thermal melting analysis; and
(d) analyzing the melting curve to determine whether the patient has a disease or disorder-causing variant.

14. The method of claim 13, wherein the target strand of a nucleic acid molecule having a locus of interest is amplified and the second genomic region is not amplified.

15. The method of claim 13, wherein the probe is unlabeled.

16. The method of claim 13, wherein the probe is blocked on its 3′ end.

17. The method of claim 13, wherein the probe has a sequence that is complementary to a wild-type sequence of the gene.

18. The method of claim 13, wherein the Tm of the probe is less than about 5° C. lower than the lowest Tm of the excess primer and the limiting primer.

19. The method of claim 13, wherein the excess primer anneals to a sequence that is unique on the target strand of a nucleic acid molecule having a locus of interest and the limiting primer anneals to a sequence that is substantially homologous to the second genomic region and set close to the variant to minimize amplicon size for high genotyping sensitivity.

20. The method of claim 13, wherein the variant is A455E of the CFTR gene.

21. The method of claim 13, wherein the variant is 1461ins4 of the CFTR gene.

22. The method of claim 20, wherein the excess primer has a nucleotide sequence of 5′-gggccatgtgcttttcaaact-3′ (SEQ ID NO:5).

23. The method of claim 20, wherein the limiting primer has a nucleotide sequence of 5′-gaactaccttgcctgctcca-3′ (SEQ ID NO:6).

24. The method of claim 20, wherein the probe has a nucleotide sequence of 5′-aaccgccaacaactgtcctctttctat-3′ (SEQ ID NO:7) and is blocked at its 3′ end.

25. A method of detecting a disease or disorder in a patient based on said patient's genotype and a priori knowledge of disease or disorder-causing variant gene sequence associated with said disease or disorder, wherein the variant gene sequence is on a target strand of a nucleic acid molecule having a locus of interest that is substantially homologous to a second genomic region, the method comprising:
(a) obtaining a biological sample from a patient, wherein the sample contains a nucleic acid molecule having the locus of interest and a second genomic region substantially homologous to the locus of interest;
(b) performing asymmetric PCR on a portion of the biological sample to produce an amplicon;
(c) subjecting the portion to an unlabeled probe assay to produce a melting curve; and
(d) analyzing the melting curve to determine whether the patient has a disease or disorder-causing variant.

26. The method of claim 25, wherein the unlabeled probe assay comprises hybridizing an unlabeled probe to a locus of interest on the amplicon to form a probe-amplicon element, adding a saturated dye to the probe-amplicon element to form a mixture, and generating a melting curve for the probe-amplicon element by measuring fluorescence from said dye as the mixture is heated.

27. The method of claim 25, wherein the target strand of a nucleic acid molecule having a locus of interest is amplified and the second genomic region is not amplified.

28. The method of claim 25, wherein the probe is unlabeled.

29. The method of claim 25, wherein the probe is blocked on its 3′ end.

30. The method of claim 25, wherein the probe has a sequence that is complementary to a wild-type sequence of the gene.

31. The method of claim 25, wherein the Tm of the probe is less than about 5° C. lower than the lowest Tm of the excess primer and the limiting primer.

32. The method of claim 25, wherein the excess primer anneals to a sequence that is unique on the target strand of a nucleic acid molecule having a locus of interest and the limiting primer anneals to a sequence that is substantially homologous to the second genomic region and set close to the variant to minimize amplicon size for high genotyping sensitivity.

33. The method of claim 25, wherein the variant is A455E of the CFTR gene.

34. The method of claim 25, wherein the variant is 1461ins4 of the CFTR gene.

35. The method of claim 33, wherein the excess primer has a nucleotide sequence of 5′-gggccatgtgcttttcaaact-3′ (SEQ ID NO:5).

36. The method of claim 33, wherein the limiting primer has a nucleotide sequence of 5′-gaactaccttgcctgctcca-3′ (SEQ ID NO:6).

37. The method of claim 33, wherein the probe has a nucleotide sequence of 5′-aaccgccaacaactgtcctctttctat-3′ (SEQ ID NO:7).

38. A kit comprising:
(a) a primer having a nucleotide sequence of 5′-gggccatgtgcttttcaaact-3′ (SEQ ID NO:5);
(b) a primer having a nucleotide sequence of 5′-gaactaccttgcctgctcca-3′ (SEQ ID NO:6);
(c) a probe having a nucleotide sequence of 5′-aaccgccaacaactgtcctctttctat-3′ (SEQ ID NO:7); and
(d) instructions for performing a diagnostic test for detecting cystic fibrosis transmembrane conductance regulator exon 9 variants using a biological sample from a patient.

39. The kit of claim 38, wherein the probe is blocked at its 3′ end.

40. A method of designing primers and probes that are useful for thermal melt analysis of a nucleic acid having a locus of interest that contains a disease or disorder-causing variant and that is substantially homologous to a second genomic region, the method comprising:
(a) selecting a locus of interest of a disease, in which the locus of interest has a disease or disorder-causing variant and in which the locus of interest is on a nucleic acid that is substantially homologous to a second genomic region;
(b) designing a pair of primers for use in asymmetric PCR using an appropriately programmed computer, wherein one primer is designed using the computer to hybridize to a unique heterologous region in the locus of interest and to not hybridize to the second genomic region and the other primer is designed using the computer to hybridize as close as possible to the variant in the locus of interest to minimize amplicon size; and
(c) designing a probe for hybridizing to one strand of the locus of interest using an appropriately programmed computer, wherein the nucleotide sequence of the probe is complementary to the nucleic acid's wild-type sequence or to the variant sequence.

41. The method of claim 40, wherein the probe is complementary to the nucleic acid's wild-type sequence.

42. The method of claim 40, wherein the primer that hybridizes to the unique heterologous region is the excess primer and the primer that hybridizes to the homologous region is the limiting primer.

43. The method of claim 40, wherein the probe is blocked at its 3′ end.

44. A method of detecting on a variant on a target strand of a nucleic acid molecule having a locus of interest that is substantially homologous to a second genomic region, the method comprising:
(a) providing an amplicon having a locus of interest, wherein the amplicon is an amplicon of the nucleic acid and is not an amplicon of the second genomic region;
(b) hybridizing an unlabeled probe to the amplicon to produce a probe-amplicon element;
(c) generating a melting curve for the probe-amplicon element in a mixture with a saturating binding dye by measuring fluorescence from the dye as the mixture is heated; and
(d) analyzing the melting curve to determine whether the variant is present in the nucleic acid.

45. The method of claim 44, wherein the probe is blocked on its 3′ end.

46. The method of claim 44, wherein the probe has a sequence that is complementary to a wild-type sequence of the gene.

47. The method of claim 44, wherein the variant is A455E of the CFTR gene.

48. The method of claim 44, wherein the variant is 1461ins4 of the CFTR gene.

49. The method of claim 47, wherein the probe has a nucleotide sequence of 5′-aaccgccaacaactgtcctctttctat-3′ (SEQ ID NO:7) and is blocked at its 3′ end.

50. A system for identifying a variant on a target strand of a nucleic acid molecule having a locus of interest that is substantially homologous to a second genomic region comprising:
(a) a microfluidic device comprising a plurality of sample loading zones, each of said sample loading zones being configured to house a separate asymmetric PCR using a nucleic acid having a locus of interest;
wherein said microfluidic device comprises at least one sample loading zone that is loaded with a limiting primer, an excess primer, and an unlabeled probe;
(b) a HRMA device, comprising a heating element, a fluorescence excitation light source and a fluorescence collection aperture, configured to thermally melt probe-amplicon elements obtained from asymmetric PCRs in said sample loading zones, and to generate fluorescence derivative melting curves for said probe-amplicon elements; and
(c) a fluorescence derivative melting curve analysis device configured to analyze the melting curves generated by said HRMA device so as to identify a variant on a target strand of a nucleic acid molecule having a locus of interest that is substantially homologous to a second genomic region.
